(19) United States
(12) Patent Application Publication
(10) Pub. No.: US 2024/0241115 A1
(43) Pub. Date: Jul. 18, 2024

(54) SYSTEM, DEVICES, AND METHODS FOR MEASURING ANTIBODY TITER AND GLYCOSYLATION

(71) Applicants: Chen Yu CHEN, College Park, MD (US); UNIVERSITY OF MARYLAND, COLLEGE PARK, College Park, MD (US)

(72) Inventors: Chen Yu CHEN, College Park, MD (US); William E. BENTLEY, Annapolis, MD (US); Dana MOTABAR, Darnestown, MD (US); Jinyang LI, Pasadena, CA (US); Sally Patricia WANG, Greenbelt, MD (US); Chen-Yu TSAO, Potomac, MD (US); Gregory F. PAYNE, Hunt Valley, MD (US)

(21) Appl. No.: 18/554,409

(22) PCT Filed: Apr. 11, 2022
(86) PCT No.: PCT/US2022/024292

§ 371 Date: Oct. 6, 2023

Related U.S. Application Data
(60) Provisional application No. 63/173,289, filed on Apr. 9, 2021.

(51) Int. Cl. G01N 33/543; G01N 27/12; G01N 33/68

(52) U.S. Cl. CPC G01N 33/5438; G01N 27/126; G01N 33/6854; G01N 2333/42

(57) ABSTRACT

The present disclosure provides sensors, devices, systems and methods for detecting an analyte in a sample using electrochemical readouts. The sensors are electroassembled with abiological recognition element and are capable of specifically binding to analytes within a sample.

### CROSS-REFERENCE TO RELATED APPLICATION(S)

This application claims the benefit of U.S. Provisional Patent Application Ser. No. 63/173,289 filed on Apr. 9, 2021, the content of which hereby is incorporated by reference herein in its entirety.

### GOVERNMENT SUPPORT

This invention was made with government support under 70NANB17H002 awarded by the National Institute of Standards and Technology, and under CBET1805274 and ECCS1809436 awarded by the National Science Foundation. The government has certain rights in the invention.

### SEQUENCE LISTING

A Sequence Listing accompanies this application and is submitted as an ASCII text file of the sequence listing named “171351_00036_ST25.txt” which is 727 bytes in size and was created on Apr. 11, 2022. The sequence listing is electronically submitted via EFS-Web with the application and is incorporated herein by reference in its entirety.

### BACKGROUND

Recombinant antibody therapeutics constitute a large segment of the biologics market as they have shown clinical and commercial success in many therapeutic areas, including cancer, respiratory and autoimmune diseases, among others. The development process for antibody therapeutics is costly and time intensive due to the complexities associated with optimizing host cell lines, bioreactors, and processing conditions, along with the post-synthesis product characterization. Reducing the cost and the time required to characterize and develop these therapeutics is important in order for biopharmaceutical companies to maintain competitiveness, reach their clinical goals, and most importantly, deliver safe and effective therapeutics for patients.

An important part of the antibody development process includes monitoring and characterizing critical process parameters (CPP) (e.g., antibody titer) and the product's critical quality attributes (CQA) (e.g., N-linked glycosylation). Monitoring and characterization of CPPs and CQAs is exceedingly important as any deviations can significantly impact the safety and efficacy of the therapeutic. Gold standard analytical tools used for monitoring and characterization can provide detailed information but are expensive, time intensive, and require specialized training (e.g., mass spectrometry, HPLC-based capillary electrophoresis).

Analytical technologies that can quickly provide estimates of CPPs and CQAs are also needed, however, as they can be integrated with several data analytic methods providing discrimination and model-based control. Simple electrochemical transduction methods are ideal for this application as they allow for rapid and sensitive measurement as well as convenient connectivity to process control systems, as has been the case for decades with electrochemical pH and dissolved oxygen (DO) probes for on-line monitoring in bioreactors.

Accordingly, there remains a need for devices and methods for analyzing and/or measuring biological molecules (e.g., proteins and antibodies) with high efficiency and at reduced cost.

### SUMMARY OF THE DISCLOSURE

In one aspect, the present disclosure provides a sensor. The sensor comprises an electrode comprising an electronically conductive surface; a polymer layer deposited on the electronically conductive surface of the electrode; and a biological recognition element attached to the polymer layer.

In another aspect, the present disclosure provides a device. The device comprises the sensor described herein and at least one additional component.

In another aspect, the disclosure provides a method of preparing a sensor. The method comprises (a) contacting an electrode comprising an electronically conductive surface with a redox mediator and a polymer in a buffer solution; (b) depositing the polymer onto the electronically conductive surface of the electrode to generate a polymer-coated electrode, wherein the polymer-coated electrode comprises a plurality of —SH groups; and (c) applying to the polymer-coated electrode a biological recognition element. In some aspects, the polymer in (a) comprises a plurality of —SH groups.

In another aspect, the disclosure provides a method of detecting and/or quantitating an analyte in a sample, comprising (a) contacting the sample with the sensor or the device described herein, wherein the analyte binds to the sensor or the sensor of the device; and (b) detecting the bound analyte from the sample on the sensor.

In yet another aspect, the disclosure provides a method of detecting and/or quantitating an antibody in a sample, comprising (a) contacting the sample with the sensor of any one of claims 1-9, wherein the biological recognition element comprises a cysteine-tagged protein G, wherein the antibody in the sample binds to the sensor; and (b) detecting the attached antibody.

In a further aspect, the disclosure provides a method of analyzing a glycosylated antibody in a sample, comprising (a) contacting the sample with the sensor of, wherein the biological recognition element comprises a thiolated sugar, optionally wherein the thiolated sugar is linked to a sugar-specific lectin, thereby attaching the glycosylated antibody in the sample to the sensor; and (b) detecting the attached glycosylated antibody.

The foregoing and other aspects and advantages of the embodiments of the present disclosure will appear from the following description. In the description, reference is made to the accompanying drawings which form a part hereof, and in which there is shown by way of illustration preferred embodiments of the disclosure. Such embodiments are illustrative only, are not intended to be limited, and do not necessarily represent the full scope of the present disclosure, however, and reference is made therefore to the claims herein for interpreting the scope of the invention. As such, features of the presently disclosed subject matter will be apparent from the following detailed description and the appended claims that follow.

### BRIEF DESCRIPTION OF THE DRAWINGS

Further objects, features and advantages of the present disclosure will become apparent from the following detailed description taken in conjunction with the accompanying figures showing illustrative embodiments of the present disclosure, in which:

FIG. 1(a) shows a representative process to construct and achieve an electrochemical response from the titer and galactosylation detection interfaces.

FIG. 1(b) shows an electron transfer mechanism to achieve an electrochemical response involved the cycling of Fc, HRP and H₂O₂.

FIG. 2(a) shows oxidation of thiolated PEG enabled the construction of the interfaces.

FIG. 2(b) shows fluorescence microscopy of FITC cysteinylated protein G with interfaces of (i) electrodeposited thiolated PEG, (ii) bare gold, and, FITC RCA120 with interfaces of (iii) electrodeposited thiolated PEG incubated in thiolated Galβ, (iv) bare gold, (v) electrodeposited thiolated PEG, and (vi) thiolated Galβ. The area of the gold surface of the electrode is 3.1 mm².

FIGS. 3(a) and 3(b) demonstrate that CV profiles and resultant bar graphs showed an increase in peak-to-peak separation (indicated by arrows) and decrease in peak current response for titer detection (FIG. 3(a)) and galactosylation detection (FIG. 3(b)) interfaces.

FIGS. 3(c) and 3(d) demonstrate titer and galactosylation detection. Corresponding Nyquist plots for titer detection (FIG. 3(c)) and galactosylation detection (FIG. 3(d)) demonstrate that layers formed on electrode surfaces as evidenced by the increase in the diameter of the semi-circular area (indicated by arrows).

FIG. 4(a) shows that the charge profiles during individual hydrogel electrodepositions were consistent.

FIG. 4(b) shows CV profiles indicating that current measurements of individually prepared thiolated PEG hydrogels were reproducible.

FIG. 4(c) shows fluorescence microscopy images and corresponding fluorescent intensity measurements of individually prepared titer detection and galactosylation detection interfaces indicating that interface construction was consistent.

FIG. 5(a) shows a schematic illustrating an experimental setup in which, upon addition of H₂O₂, an electrochemical response that can be correlated to antibody concentration is achieved.

FIG. 5(b) shows a representative current responses from each IgG concentration evaluated by the titer detection interface.

FIG. 5(c) shows a linear concentration dependent response, ranging from 15.6-1000 μg/mL of IgG, was achieved. Each point represents the average current response from 4 individual titer detection interfaces.

FIG. 6(a) shows a schematic describing an experimental setup for galactosylation detection which is similar to that of the titer detection interface.

FIG. 6(b) shows antibody glycoforms used to characterize the interface above each corresponding bar in the graph. Each bar represents the average current response from 4 individual galactosylation detection interfaces. Statistical significance (p<0.005) was determined between groups* and** using ANOVA: Single Factor analysis.

FIG. 7(a) shows evaluation of titer detection with IgG spiked in PBS with 0.05% Tween-20 or in DMEM.

FIG. 7(b) shows normalized values for all titer detection interface measurements.

FIG. 7(c) shows galactosylation detection interfaces with IgG spiked in PBS or in DMEM. Each point and bar represent the average current response from 3 individual interfaces.

FIG. 8(a) shows an experimental protocol for evaluation of the regeneration of the titer detection interface (top) and results for titer detection interfaces regenerated for reuse (bottom).

FIG. 8(b) shows an identical protocol as in FIG. 8(a) for regeneration of the galactosylation detection interface (top) and results for galactosylation detection interfaces regenerated for reuse (bottom). Each bar represents the average current responses from 3 individual interfaces.

FIG. 9(a) shows the plasmid map of a representative cysteinylated protein G.

FIG. 9(b) shows a Western Blot confirming the expression of the cysteinylated protein G.

FIG. 10 shows controls for the titer detection interface include interfaces assembled without protein G:HRP (1 g/L IgG only), without IgG (0.1 g/L protein G:HRP only), and without IgG and protein G:HRP. Each bar represents the average current response from 4 individual interfaces.

FIG. 11 shows description of the N-linked glycan patterns of the antibody glycoforms used to evaluate the galactosylation detection interface.

FIG. 12(a) shows a schematic depicting the experimental protocol for the titer detection interface (top). After initial incubation and measurement, interfaces were washed in regeneration buffer and another measurement was taken. Interfaces were then re-incubated in protein G:HRP (0.1 g/L) only. The current response indicates that IgG has been removed from the interface after regeneration washes (bottom).

FIG. 12(b) shows a schematic depicting the experimental protocol for the galactosylation detection interface (top). Similar to the titer detection interface, IgG has been removed from the interface after regeneration washes. Each bar represents the average current response from 3 individual interfaces.

FIG. 13(a) shows fluorescence microscopy images indicating that FITC RCA120 is still bound to the interface after the regeneration washes. Fluorescence microscopy images were then taken with an exposure of 50 ms.

FIG. 13(b) shows CV profile (left) and corresponding Nyquist plot (right) indicating that the RCA120 can bind directly to the thiolated PEG hydrogel when thiolated Galβ is not present.

FIG. 14 demonstrates thiolated fluorescent molecule and Cys-tagged fluorescent protein can be conjugated to PEG-SH gels through disulfide bond, respectively. Experimentally, PEG-SH gels (50 mg/mL with 5 mM Fc, +0.5 V) were electrodeposited on electrodes for 5 mins, followed by over-oxidization (i.e., activation) in solutions containing 5 mM Fc at +0.5 V for 5 min, to ensure the oxidation of all thiol groups. As illustrated by FIG. 14, PEG-SH gels were then exposed to solutions containing FITC-PEG-SH (50 μg/mL) or Cys-DsRed (200 μg/mL) for 2 hrs, followed by a fluorescence microscopy imaging. The results show the spatially-selective deposition and modification of PEG-SH gels is independent of electrode materials, and show the electrodeposited PEG-SH gel facilitates efficient assembly of Cys-DsRed at reduced state.

### DETAILED DESCRIPTION

The present disclosure relates to sensors, devices, systems, and methods for analyzing biomolecules. In particular, the present sensors, devices, systems, and methods may be used to measure antibody titer and glycosylation. In certain embodiments, the systems and devices include a sensor with a detection interface that is capable of measuring the amount of the protein within the sample, for example, capable of measuring antibody titer and/or glycosylation through the transduction of an electrochemical output. In certain embodiments, the detection interface makes use of a PEG thiol-mediated assembly methodology and may be rapidly electroassembled onto sensor electrodes. In certain embodiments, the detection interface comprises a cysteine-tagged protein that is capable of recognizing an analyte (e.g., protein) within a sample. The analyte can be detected as to binding to the recognition element, and in some embodiments, further quantitated (e.g., protein concentration). In some embodiment, the recognition element is a cysteine-tagged protein G antibody recognition protein and may be used to detect antibody titer.

For example, the cysteine-tagged protein G antibody recognition protein may be linked to the thiolated PEG interface, which may enable rapid, robust assessment of total antibody concentration. In certain embodiments, the detection interface comprises thiolated sugar groups linked to thiol-PEG binding sites so that the sugar groups act as a lectin bait, which may be used to detect glycosylation. For example, sugar-specific lectins may be layered on top of the thiolated PEG hydrogel, and these surfaces may provide selective capture of antibodies based on the lectin-glycan binding. In certain embodiments, the present detection interfaces may be coupled with an electrochemical reporter, enabling near real time electrochemical outputs.

### Definitions

Unless otherwise defined, all technical and scientific terms used herein have the same meaning as commonly understood by one skilled in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987; the entire contents of each of which are incorporated herein by reference.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Sensors

The present disclosure provides sensor interfaces that may be used, for example, for protein detection, including antibody detection, antibody titer and N-linked galactosylation detection. The sensor interfaces may produce electrochemical outputs from electrodes upon which the sensing domains are also electroassembled. Unlike traditional analytical chemistry methods, the present sensor interfaces may couple electrochemical techniques with biological molecular recognition elements.

In one aspect, provided is a sensor comprising: an electrode comprising an electronically conductive surface; a polymer layer deposited on the electronically conductive surface of the electrode; and a biological recognition element attached to the polymer layer.

The electrode may comprise any suitable electrical conductor material for use in an electrical circuit and is not limited to any particular shape or size. An electric potential or current may be applied to the electrode. The electronically conductive surface may comprise an electronically conductive material. Suitable electronically conductive materials include, but are not limited to, metals (such as gold, indium, copper, silver, cobalt, nickel, platinum, etc.), metal oxides (such as indium tin oxide (ITO), tin oxide, zinc oxide, etc.), conductive glasses (such as metal or metal oxide coated glass, e.g., indium tin oxide coated glass, fluorine doped tin oxide coated glass, molybdenum (Mo) coated glass, and aluminum doped zinc oxide (AZO) coated glass), conductive carbon materials (such as carbon nanotubes (CNT), graphene, nanowire meshes, etc.), or a combination thereof.

In some embodiments, the electronically conductive surface comprises a metal, a metal oxide, a conductive glass, and/or a conductive carbon material. In some embodiments, the electronically conductive surface comprises gold, such as a gold coating or a gold layer on the electrode.

The electronically conductive surface is not limited to any particular shape, size (e.g., area, length, width, diameter), or thickness. The thickness of the electronically conductive surface may be adjusted according to the analytical methods as disclosed herein. In some embodiments, the thickness may range from nanometers (nm) to micrometers (m) or millimeters (mm). For example, the thickness can be about 10 nm, about 100 nm, about 1 m, about 10 m, about 100 m, or about 1 mm. In some embodiments, the electronically conductive surface has an area of about 0.1 mm² to about 100 mm². For example, the area may be about 0.5 mm², about 1 mm², about 3 mm², about 5 mm², about 10 mm², about 20 mm², about 50 mm², or about 80 mm².

The electronically conductive surface may be a patterned surface and/or a surface laminated onto the electrode according to the analytical methods as disclosed herein. In some embodiments, the electronically conductive surface is a patterned surface. In some embodiments, the electronically conductive surface may be arranged into an array of multiple individual surfaces, each of which may be used or controlled separately.

The sensor comprises a polymer layer deposited on the electronically conductive surface of the electrode. The polymer can be thiolated polymers or hydrophilic polymers that are able to be crosslinked and form a layer on the electrode. Suitable polymers include, for example, one or more of hyaluronic acid, polyethylene glycol (PEG), polypropylene glycol, polyglutamate, polylysine, polysialic acid, polyvinyl alcohol, polyacrylate, polymethacrylate, polyacrylamide, polysaccharide, among others, and these polymers, in some embodiments, are thiolated to allow for crosslinking of the polymers. In some embodiments, the polymer layer comprises a biopolymer, such as a sugar (e.g., chitosan), a protein (e.g., gelatin, collagen, fibrin), or an engineered protein (e.g., a protein with fusion tags including, for example, cysteine residues). In some embodiments, the polymer comprises a thiolated biopolymer, e.g., a thiolated protein or engineered protein that comprises a cysteine capable of forming a disulfide bond. Polymers can be thiolated by the immobilization of sulfhydryl ligands within the polymer or the substitution of hydroxyl moieties on polymers by thiol groups. In some embodiments, the polymer comprises a protein with a fusion tag containing cysteine residues.

In some embodiments, the polymer layer comprises a hydrogel. The hydrogel may be a polymeric material having a network of hydrophilic polymers. The hydrophilic polymers may be natural or synthetic polymers. Suitable hydrogels include hydrogels comprising one or more of hyaluronic acid, polyethylene glycol (PEG), polypropylene glycol, polyglutamate, polylysine, polysialic acid, polyvinyl alcohol, polyacrylate, polymethacrylate, polyacrylamide, and polysaccharide. In some embodiments, the hydrogel comprises gelatin, collagen, or chitosan. In some embodiments, the formed hydrogel is thiolated in order to bind to the biological recognition element. In a preferred embodiment, the polymer contains a thiol (e.g., thiolated PEGs or proteins).

The hydrogel may be produced, for example, by crosslinking the polymer molecules. In some embodiments, the polymers comprise a plurality of sulfhydryl groups (or thiol groups, —SH) and the polymers may be crosslinked by forming disulfide (S—S) bond between polymer molecules. In some embodiments, the hydrogel comprises a network of polymers crosslinked by S—S bonds. Suitable thiol-functionalized polymers for forming hydrogels crosslinked by S—S bonds include, for example, thiolated polyethylene glycol (PEG), thiolated polypropylene glycol, thiolated polyglutamate, thiolated polylysine, thiolated polysialic acid, thiolated polyvinyl alcohol, thiolated polyacrylate, thiolated polyacrylamide, and thiolated polysaccharides. The polymers (e.g., PEG polymers) forming the present polymer layer are not limited to any particular molecular weight. In some embodiments, the polymer layer (e.g., a hydrogel) further comprises a thiolated linker molecule. For instance, a hydrogel (e.g., a gelatin, collagen, or chitosan hydrogel) may be formed, and a cystine-containing molecule (e.g., a protein or engineered protein) or another thiolated molecule may be attached (e.g., grafted) to the hydrogel.

In some embodiments, the polymer layer comprises thiolated polyethylene glycol (PEG). For example, the thiolated PEG polymer is a multiarmed polymer, such as a 4-arm or an 8-arm polymer. In some embodiments, the polymer is a 4-arm thiolated PEG polymer. For example, the 4-arm thiolated PEG polymer may have a structure shown below, with a thiol group at each terminus of the four PEG arms connected to a pentaerythritol core. The 4-arm thiolated PEG polymer may have a molecular weight of about 1 kDa to about 10 kDa, such as about 2 kDa or about 5 kDa.

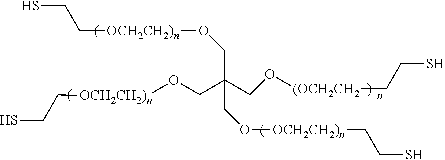

In some embodiments, the polymer layer is electrodeposited on the electronically conductive surface of the electrode. For example, the polymer (e.g. a PEG polymer) may form a layer, a coating, or a hydrogel that is deposited on the electronically conductive surface (e.g., a gold surface) when an electric potential is applied to the electrode. In some embodiments, the polymer is crosslinked to provide a hydrogel during the electrodeposition process. In some embodiments, the polymer is a thiolated polymer (e.g., thiolated PEG), and the thiol groups of thiolated polymer molecules form S—S bonds during electrodeposition, thus providing a crosslinked polymer layer (e.g., hydrogel) on the electronically conductive surface of the electrode. The thiol groups and/or the S—S bonds in the polymer layer may bind to the electronically conductive surface (e.g., gold or silver surface) such that the polymer layer is deposited on the electronically conductive surface.

The biological recognition element may be attached to the polymer layer, for example by chemical bonds or by affinity binding. In some embodiments, the biological recognition element is attached to the polymer layer by forming chemical bonds. The chemical bond may be an ionic bond, a covalent bond, a dipole-dipole interaction, or a hydrogen bond. In some embodiments, the biological recognition element is attached to the polymer layer by a disulfide bond (S—S), e.g., by reacting the additional —SH in the thiolated polymer (or the thiol groups of the linker molecules) with cysteines within the biological recognition element (e.g., protein).

The biological recognition element may comprise, for example, a ligand (e.g. a small molecule ligand), an enzyme substrate, a cofactor, a peptide, a protein, a nucleic acid (e.g., DNA or RNA), a sugar molecule, or a combination thereof. The biological recognition element may function as a probe, or form a functionalized interface, that detects an analyte molecule (e.g., through specific binding) in a sample. For attachment to the polymer layer, the biological recognition element may be functionalized, for example, by a thiol group.

In one embodiment, the biological recognition element is a peptide or protein that is capable of specifically binding to the analyte to be detected. The terms “protein,” “peptide,” and “polypeptide” are used interchangeably herein and refer to a polymer of amino acid residues connected to the other by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to any amino acid sequence.

Specifically, in some embodiments, the protein or peptide comprises one or more cysteine which is capable of forming a disulfide bond with the electrode and capable of binding to an analyte in the sample. In some embodiments, the protein or peptide can be cysteine-tagged.

In one embodiment, biological recognition element comprising the peptides or proteins are antibodies or antigen-binding fragments thereof. Suitably, the antibody is capable of specifically binding to the analyte to be detected in the sample.

In some embodiments, the biological recognition element comprises a peptide or protein capable of antibody detection. For example, the biological recognition element may comprise protein G, which possesses high binding affinity for the Fc domain of immunoglobulin G (IgG). For attachment to the polymer layer, the protein G may be a cysteine-tagged protein G.

In some embodiments, the biological recognition element comprises a sugar molecule for detecting biomolecules that interact, directly or indirectly, with saccharides. For attachment to the polymer layer, the sugar molecule may be a thiolated sugar. The thiolated sugar may comprise, for example, thiolated monosaccharides, thiolated disaccharides, thiolated oligosaccharides, or thiolated polysaccharides. The sugar of the thiolated sugar may comprise, for example, glucose, mannose, galactose, fructose, xylose, sucrose, lactose, maltose, isomaltulose, trehalose, or a derivative thereof.

In some embodiments, the thiolated sugar is also linked (e.g., by specific binding) to a saccharide binding protein, such as a sugar-specific lectin, for detecting biomolecules that specifically bind to such saccharide binding protein. The lectin may be, for example, a mannose-binding lectin (e.g., concanavalin A (Canavalia ensiformis), lentil lectin (Lens culinaris), snowdrop lectin (Galanthus nivalis)), a galactose/N-acetylgalactosamine binding lectin (e.g., RCA120 (Ricinus communis), peanut agglutinin (Arachis hypogaea), jacalin (Artocarpus integrifolia), hairy vetch lectin (Vicia villosa)), a N-acetylglucosamine binding lectin (e.g., wheat germ agglutinin (Triticum vulgaris)), a N-acetylneuraminic acid binding lectin (e.g., elderberry lectin (Sambucus nigra), Maackia amurensis leucoagglutinin (Maackia amurensis), Maackia amurensis hemaagglutinin (Maackia amurensis)), or a fucose binding lectin (e.g., Ulex europaeus agglutinin (Ulex europaeus), Aleuria aurantia lectin (Aleuria aurantia)). In some embodiments, the sugar-specific lectin is RCA120.

In some embodiments, the biological recognition element comprises a cysteine-tagged protein G linked to the polymer layer, or a thiolated sugar linked to the polymer layer. The thiolated sugar may be further linked to a sugar-specific lectin. In some embodiments, the biological recognition element comprises the thiolated sugar, wherein the thiolated sugar is deposited on the polymer layer (e.g., by S—S bonds) and the sugar-specific lectin is deposited on top of the thiolated sugar. For example, the thiolated sugar may comprise a thiolated saccharide. The thiolated saccharide may be a thiolated monosaccharide, such as thiolated Galβ, or 2-thioethyl β-galactopyranoside.

The analyte can be any molecule within a sample that specifically binds to the biological recognition elements and is of interest to be detected and/or quantified. Suitable analytes include, ligands, proteins, peptides, complexes, glycosylated proteins. In one embodiment, the analytes are proteins, and include, for example, antibodies.

### Devices and Systems

In another aspect, the present disclosure provides a device comprising the sensor as described herein and at least one additional component. The device is capable of detecting an analyte in a sample. The at least one additional component may comprise, for example, a housing, a counter electrode, a reference electrode, a computer, a data storage unit, a controlling unit, a power supply, or combinations thereof. In some embodiments, the device comprises the sensor as a working electrode, a counter electrode, and a reference electrode. The power supply is capable of providing an electric potential across the electrode.

In some embodiments, the device comprises an array of a plurality of the sensors as described herein. The array of sensors may be used to detect multiple analytes, and output of each sensor may be monitored individually. In another embodiment, the array of sensors may be used to detect the same analyte in multiple samples.

In another aspect, the present disclosure also provides an electrochemical instrument or analytical system that comprises the device as disclosed herein.

### Sensor Preparation

Methods of preparing the sensors described herein are provided. While the examples demonstrate a preferred embodiment of methods of preparation, other methods of preparing sensors are within the scope that provide the sensors described herein. In some embodiments, the sensors are prepared by electrodepositing the polymer onto the electrode surface. Suitable methods are known in the art.

In one embodiment, provided is a method of preparing a sensor, comprising: (a) contacting an electrode comprising an electronically conductive surface with a redox mediator and a polymer in a buffer solution; (b) depositing the polymer onto the electronically conductive surface of the electrode to generate a polymer-coated electrode, wherein the polymer-coated electrode comprises a plurality of —SH groups; and (c) applying to the polymer-coated electrode a biological recognition element. In some embodiments, the polymer in (a) comprises a plurality of —SH groups. In some embodiments, the deposition process in (b) comprises applying an electric potential to the electrode, thereby electrodepositing the polymer onto the electronically conductive surface of the electrode. In some embodiments, the deposition process in (b) comprises (b-i) depositing the polymer onto the electronically conductive surface of the electrode; optionally wherein the deposited polymer are crosslinked to form a hydrogel; and (b-ii) attaching a plurality of thiolated linker molecules to the deposited polymers to generate the polymer-coated electrode.

In another embodiment, provided is a method of preparing a sensor, comprising: (a) contacting an electrode comprising an electronically conductive surface with a redox mediator and a polymer comprising a plurality of —SH groups in a buffer solution; (b) applying an electric potential to the electrode, thereby electrodepositing the polymer onto the electronically conductive surface of the electrode to generate a polymer-coated electrode; and (c) applying to the polymer-coated electrode a biological recognition element.

The preparation method may be used to produce a sensor as described herein. For example, the electronically conductive surface may comprise a metal, a metal oxide, a conductive glass, a conductive carbon material, or a combination thereof. In some embodiments, the electronically conductive surface comprises gold.

The polymer comprising a plurality of —SH groups may comprise, for example, a thiolated polyethylene glycol (PEG), a thiolated polypropylene glycol, a thiolated polyglutamate, a thiolated polylysine, a thiolated polysialic acid, a thiolated polyvinyl alcohol, a thiolated polyacrylate, a thiolated polyacrylamide, a thiolated polysaccharides, or a combination thereof. In some embodiments, the polymer comprises thiolated polyethylene glycol (PEG), such as a 4-arm or an 8-arm thiolated PEG. In some embodiments, the polymer comprises a biopolymer, such as a sugar (e.g., chitosan), a protein (e.g., gelatin, collagen, fibrin), or an engineered protein (e.g., a protein with fusion tags). In some embodiments, the preparation method comprises depositing a biopolymer (e.g., gelatin, collagen, or chitosan), followed by attaching a thiolated linker molecule (e.g., a cystine-containing protein or engineered protein) to the biopolymer.

The potential for electrodeposition may be, for example, an oxidative potential. The potential may be about 0 V to about 10 V. The potential may be at least 0.01 V, at least 0.1 V, at least 0.2 V, at least 0.3 V, at least 0.4 V, at least 0.5 V, at least 0.6 V, at least 0.7 V, at least 0.8 V, at least 0.9 V, at least 1 V, at least 2 V, at least 3 V, at least 4 V, at least 5 V, at least 6 V, at least 7 V, at least 8 V, or at least 9 V. The potential may be at most 10 V, at most 9 V, at most 8 V, at most 7 V, at most 6 V, at most 5 V, at most 4 V, at most 3 V, at most 2 V, at most 1 V, at most 0.9 V, at most 0.8 V, at most 0.7 V, at most 0.6 V, at most 0.5 V, at most 0.4 V, at most 0.3 V, at most 0.2 V, or at most 0.1 V. In some embodiments, the potential is about 0.1 V to about 1 V, such as about 0.2 V, about 0.3 V, about 0.4 V, about 0.5 V, or about 0.8V.

In some embodiments, the deposition process in (b) comprises crosslinking the polymer. The crosslinked polymer, for example, may form a hydrogel. In some embodiments, electrodepositing the polymer (e.g., a thiolated PEG) onto the electronically conductive surface (e.g., a gold surface) of the electrode in (b) comprises crosslinking the polymer by forming a disulfide bond (S—S) between the —SH groups. In some embodiments, the polymer crosslinked by the S—S bond forms a hydrogel.

In some embodiments, the deposition process of (b) further comprises converting a plurality of —SH groups of the polymer-coated electrode to —SOH groups (sulfenic acid groups) by the applied electric potential. For example, a plurality of surface —SH groups may remain after electrodeposition and crosslinking of the polymer on the electronically conductive surface of the electrode. Under the applied potential (e.g., an oxidative potential), the remaining —SH groups may be converted to sulfenic acid groups (—SOH) for further conjugation or attachment of the biological recognition element. In particular, the sulfenic acid groups may function as an “activated” form of the —SH groups on the surface of the polymer-coated electrode, which may readily form disulfide linkages with thiol groups from the biological recognition element.

The biological recognition element may comprise, for example, a cysteine-tagged protein G, or a thiolated sugar as disclosed herein. The thiolated sugar may be further linked to a saccharide binding protein, such as a sugar-specific lectin. In some embodiments, the biological recognition element is attached to the polymer-coated electrode by a disulfide bond (S—S).

The redox mediator may comprise any chemical agent with electrochemical activity. The redox mediator may be capable of exchanging electrons with other reagents in a redox cycling reaction in a catalytic or biological process (e.g., one mediated by an enzyme). For example, the redox mediator may exchange electrons with an oxidant (e.g., at the reaction sites of a catalyst or enzyme) or be able to diffuse to the surface of an electrode and exchange electrons there. In a repeated, cycling process, the redox mediator may function as an electron shuttle between a reagent, a biocatalyst, and an electrode. Suitable redox mediators include, but are not limited to, iridium, ferrocene or a derivative thereof, ferricyanide, ruthenium, osmium, rhodium, copper, cobalt, nickel, chromium, platinum and palladium, redox-active organic molecules such as phenolics (e.g., acetosyringone), heterophenols (e.g., aminophenols and chlorophenolindophenols), phenazines (e.g., pyocyanin), organosulfur compounds (e.g., tetrathiafulvalene and methylene blue), and radical precursors (e.g., viologens or 2,2′-azino-bis(3-ethylbenzothiazoline-6-sulphonic acid), and combinations thereof. In some embodiments, the redox mediator comprises ferrocene or a derivative thereof.

The buffer solution may be an aqueous solution comprising a buffering agent suitable for the electrodeposition process. Suitable buffering agents include, but are not limited to, phosphate, citrate, Tris, HEPES, TES, MOPS, and MES. In some embodiments, the buffer solution is a phosphate buffer solution. The buffer solution may have a pH of about 6 to about 8, such as about 6.5, about 7.0, or about 7.5.

Upon application to the polymer-coated electrode, the biological recognition element may be attached to the electrode surface in one or more reactions. In some embodiments, step (c) of the present sensor preparation method comprises immersing the polymer-coated electrode in a solution comprising a cysteine-tagged biological recognition element (e.g. cysteine containing or cysteine-tagged protein) for a sufficient time to generate a coated electrode. The cysteines within the biological recognition element may form an S—S bonds, for example, with the sulfenic acid groups (—SOH) on the surface of the polymer coated electrode thus coating or covering the polymer surface of the electrode with the biological recognition element providing the sensor. This sensor is then capable of binding its biological recognition element specifically with analytes in a sample with specificity, and the sensor using electrochemical transduction can be used detect the analyte, to determine the concentration of the analyte within the sample and/or the glycosylation state of the analyte in the case of glycosylated proteins.

In one example, the step (c) of preparing the sensor comprises immersing the polymer-coated electrode in a solution comprising a cysteine-tagged protein, e.g., cysteine-tagged protein G, for a sufficient time to generate a protein-coated (e.g., protein G-coated) electrode. The protein G-coated electrode may be capable of detecting or quantitating an antibody in a sample, for example, a blood sample. In some embodiments, the protein G-coated electrode is capable of determining antibody titers within the sample.

In some embodiment, step (c) of the present sensor preparation method comprises immersing the polymer-coated electrode into a solution comprising thiolated sugar for a sufficient time to generate a sugar-coated electrode. The thiolated sugar, for example, may form S—S bond with the —SOH on the surface of the polymer-coated electrode, thereby generating a sugar-coated electrode. The sugar attached to the electrode surface may be further functionalized by attaching a saccharide binding protein, such as a sugar-specific lectin. In some embodiments, step (c) further comprises subsequently immersing the sugar-coated electrode in a solution comprising a sugar-specific lectin (e.g., but not limited to, RCA120) for a sufficient time to generate a lectin-coated electrode. The sugar-coated electrode and/or the lectin-coated electrode may be used to capture, detect, measure an analyte (e.g., a protein or antibody) that specifically binds to the sugar or lectin on the surface of the electrode. In some embodiments, the sugar-coated electrode and/or the lectin-coated electrode may be capable of antibody capture.

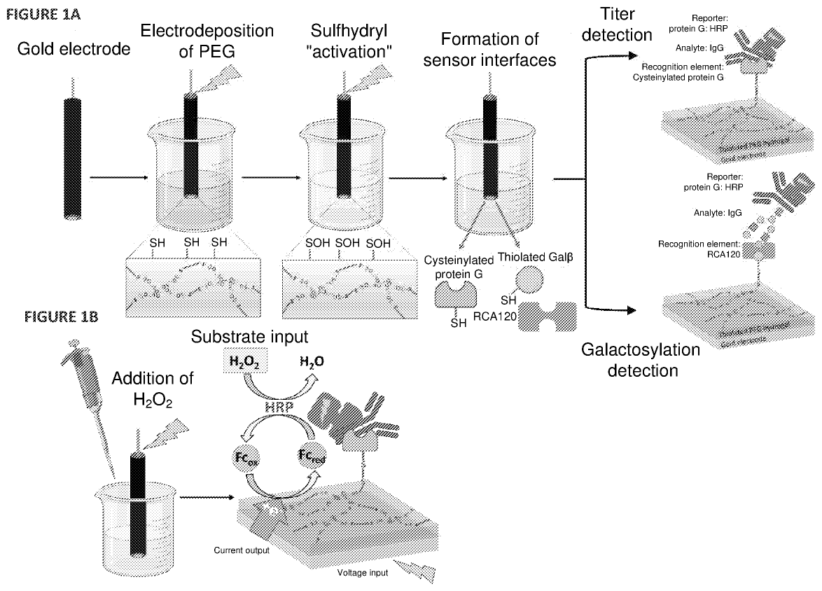

In some embodiments, a representative sensor interface may be assembled by electrodepositing a thiolated polyethylene glycol (PEG) hydrogel film on an electrode (FIG. 1A). Unlike traditional analytical chemistry methods, the sensors herein couple electrochemical techniques with biological molecular recognition elements. For example, the electrode is immersed in a solution of thiolated PEG and a redox mediator, ferrocene (Fc), at pH 7 (e.g., for 1 minute). The film thickness may be modified based on the time over which the electroassembly takes place. After electrodeposition, the polymer-coated electrode may be submerged in Fc solution and further oxidized (e.g., for 2 minutes) in order to “activate” sulfhydryl groups to reactive sulfenic acid groups (SOH as indicated). The “activated” hydrogels then may be linked with either thiolated sugars (for assembly of glycan-recognizing lectins) or a cysteine-tagged proteins (e.g., protein G). The sulfenic acid groups of the PEG may spontaneously form disulfide linkages with thiols of sugars or exposed cysteine residues cloned into proteins at their termini. In this way, molecular recognition elements may be covalently grafted onto the PEG, which in turn, may be electroassembled directly onto the sensing electrodes. PEG may be a suitable material for the interfaces as it may enable the covalent linkage of the biological recognition elements and it may provide anti-fouling properties that reduce non-specific binding. However, other suitable polymers are contemplated.

### METHOD OF USE

In another aspect, the present disclosure provides a method of detecting and/or quantitating an analyte in a sample, comprising (a) contacting the sample with the sensor or device as described herein, wherein the analyte binds to the sensor or the sensor of the device; and (b) detecting the attached analyte.

Suitable samples can be any sample in which an analyte is to be detected. The sample may be a biological sample, an environmental sample, a forensic sample, or an industrial sample. The sample may be in the form of a solution, such as an aqueous solution. For example, the sample may be a biological sample derived from a cell line, a tissue, or a body fluid (such as blood, plasma, saliva, urine, mucus, semen, vaginal fluids). In some embodiments, the biological sample may be derived from a tissue sample and made into a liquid or suspension, for example, may be a lysate from cells or tissue samples, or may be a centrifuged biological sample (e.g. blood sample). The sample may be derived from an animal or a human. The sample may be a disease sample, such as a sample derived from a human having the disease or an animal model of the disease. The disease may be, for example, cancer, metabolic disease, cardiovascular disease, respiratory disease, infectious disease. In some embodiments, the sample is derived from a cell line, such as cultured cells. In some embodiments, the sample is derived from a mammalian cell line, including cultured mammalian cell lines. For example, the sample may comprise intact or lysed cells.

The analyte may comprise a biomolecule, such as a peptide, a protein (such as an antibody), a nucleic acid, an oligosaccharide, a lipid, or a combination thereof. The analyte may bind to the biological recognition element of the present sensor. For example, the analyte and the biological recognition element may be members of a known specific binding pair (such as ligand-protein, protein-antibody, or enzyme-substrate pairs). In some embodiments, the analyte is an antibody or antigen-binding fragment thereof. For example, the antibody is an immunoglobulin G (IgG) antibody. In some embodiments, the antibody is a glycosylated antibody.

The methods described herein can be used in the development of antibodies. An important part of the antibody development process includes monitoring and characterizing critical process parameters (CPP) (e.g., antibody titer) and the product's critical quality attributes (CQA) (e.g., N-linked glycosylation). Monitoring and characterization of CPPs and CQAs is exceedingly important as any deviations can significantly impact the safety and efficacy of the therapeutic. The present methods can quickly provide estimates of CPPs and CQAs using simple electrochemical transduction methods for rapid and sensitive measurement as well as convenient connectivity to process control systems.

In some embodiments, the biological recognition element of the sensor comprises a cysteinylated protein, e.g., protein G, which may be used to detect or analyze biomolecules (e.g., antibodies) that specifically bind to protein (e.g., protein G). In some embodiments, the biological recognition element of the sensor comprises a thiolated saccharide. The thiolated saccharide may be, for example, a thiolated monosaccharide. In some embodiments, the biological recognition element comprises a thiolated saccharide linked to a saccharide binding protein. For example, the saccharide binding protein may be a lectin. In some embodiments, the lectin is RCA120. The sensor functionalized with thiolated saccharide or lectin as disclosed herein may be used, for example, to detect or analyze biomolecules (e.g., glycosylated antibodies) that specifically bind to saccharides or lectins.

In some embodiments, detecting the attached analyte comprises measuring an electrochemical response of the electrode. The electrochemical response may be an output of the sensor (e.g., electric potential or current) that can be measured using known techniques.

In some embodiments, detecting the attached analyte (e.g., protein or antibody) comprises measuring an electrochemical response of the electrode in a redox cycling reaction. For example, an attached antibody may be further coupled to a protein G conjugated to horseradish peroxidase reporter (protein G:HRP), which provides electrochemical outputs in an electron transfer process in the presence of a redox mediator (e.g., ferrocene) and hydrogen peroxide.

In some embodiments, measuring the electrochemical response of the electrode comprises measuring cyclic voltammetry (CV) and/or electrochemical impedance spectroscopy (EIS). The measurement of these electrochemical responses may be carried out using known instruments and techniques.

In some embodiments, detecting the attached analyte comprises quantitating the attached analyte. For example, the electrochemical response of the sensor electrode may be correlated with an amount of the analyte (e.g., antibody) detected by the sensor. In some embodiments, the electrochemical response of the sensor correlates linearly with the concentration of the analyte. Based on this linear relation, the concentration of the analyte in a sample may be determined, for example, by a standard curve. The concentration range of an analyte (e.g., antibody) measurable by the linear correlation to the electrochemical response of the sensor may vary in different embodiments.

Thus, the present method may be used to determine the concentration of an antibody (titer) in a sample, such as a sample derived from an antibody-producing cell line. In some embodiments, the antibody concentrate may be about 0.1 μg/mL to about 1000 μg/mL, such as about 0.5 μg/mL, about 1 μg/mL, about 5 μg/mL, about 10 μg/mL, about 50 μg/mL, about 100 μg/mL, or about 500 μg/mL.

In some embodiments, the present method comprises removing the attached analyte following detection of the analyte to form a regenerated sensor. The analyte (e.g., antibody) may be removed, for example under an acidic condition (e.g. pH 1-3). In some embodiments, the removal of the attached analyte does not disrupt or change the attachment (e.g., S—S bond) of the biological recognition element to the polymer layer (e.g., hydrogel) of the sensor. Following the removal of the analyte, the biological recognition element of the regenerated sensor may again be exposed for repeated use. Remarkably, the regenerated sensor may retain a capacity to capture the analyte that is substantially the same as that of a freshly prepared sensor. For example, the regenerated sensor may produce a response (e.g, an electrochemical response as disclosed herein) toward an analyte that is at least 50%, at least 60%, at least 70%, at least 80%, at least 85%, at least 90%, at least 95%, at least 99%, or at least 100% of the response produced by the freshly prepared sensor toward the same analyte in the sample. Thus, the present sensors and methods may provide improved efficiency of sample analysis at reduced cost.

In some embodiments, provided is a method of detecting and/or quantitating an antibody in a sample, comprising (a) contacting the sample with a sensor as described herein, wherein the biological recognition element comprises a cysteine-tagged protein G, thereby attaching the antibody in the sample to the sensor; and (b) detecting the attached antibody.

For example, the antibody in the sample (e.g., an IgG antibody) may specifically bind to the protein G on the surface of the sensor. The method may be used, for example, to determine the concentration (titer) of the antibody.

In some embodiments, provided is a method analyzing a glycosylated antibody in a sample, comprising (a) contacting the sample with a sensor as described herein, wherein the biological recognition element comprises a thiolated sugar, optionally wherein the thiolated sugar is linked to a sugar-specific lectin, thereby attaching the glycosylated antibody in the sample to the sensor; and (b) detecting the attached glycosylated antibody.

The sample (e.g., from a cell line) may comprise different antibody glycoforms, as a result of different glycosylation of the antibody. For example, glycosylation of the IgG Fc (crystallizable fragment) region is essential for the antibody's functions. Multiple glycoforms of the antibody (resulting from a heterogeneity in the oligosaccharides that are attached) can differ from each other in biological efficacy. Antibody glycosylation can also regulate antibody stability, secretion, immunogenicity, and function. The antibody glycoforms may bind to the biological recognition element of the present sensor (e.g., through binding to the lectin) with different specificity. Thus, the present sensor may be used to detect or differentiate antibodies with a specific glycosylation pattern.

In some embodiments, the present method further comprises determining a glycan structure of the glycosylated antibody. The glycan structure may include a glycan pattern. For example, the present method may be used to determine N-linked glycan patterns of antibody glycoforms (e.g., N-linked galactosylation). The present method may be implemented with an array of lectins for high throughput analysis of glycosylated antibodies. For example, the method may employ an array of the sensors as described herein each comprising a specific lectin.

In particular embodiments, for antibody titer detection, cysteinylated protein G construct may be layered onto a PEG polymer layer as disclosed herein, where it may enable total antibody titer detection via binding interactions with the antibody's fragment crystallizable (Fc) region. For antibody glycosylation detection, instead of assembling the cysteinylated protein G, thiolated sugar groups (e.g., β-galactose, Galβ) may be applied to occupy many of the open thiol-PEG binding sites so that the sugar groups may act as a lectin bait. Then, sugar-specific lectins (e.g. Ricinus communis agglutinin I, RCA120) may be layered on top of the galactosylated and thiolated PEG hydrogel. These surfaces then may provide selective capture of antibodies based on lectin-glycan binding. Further, when coupled with a protein G conjugated to horseradish peroxidase reporter (protein G:HRP), these interfaces may enable near real time electrochemical outputs, based upon, for example, the electron transfer mechanism between HRP, ferrocene, and hydrogen peroxide (H₂O₂) as shown in FIG. 1B). As depicted, the same electrodes used to assemble the sensing components may be used for electrochemical detection of the antibodies. As such, there are many advantages associated with the coupling of electroassembly and electrochemical readouts, a most simple example being that the size of the electrode may be used to vary the sensor binding capacity and signal strength. The present sensors, devices, and methodology may provide a simple, portable, inexpensive solution that yields fast and quantifiable results for a variety of applications, including bioprocessing applications.

Process conditions established during the development and manufacture of recombinant protein therapeutics dramatically impact their quality and clinical efficacy. Technologies that enable rapid assessment of product quality are critically important. The present disclosure provides sensor interfaces that directly connect to electronics and enable near real time assessment of antibody titer and N-linked galactosylation. In particular embodiments, the present disclosure provides a spatially resolved electroassembled thiolated PEG hydrogel that enables electroactivated disulfide linkages. For titer assessment, a cysteinylated protein G may be linked to the thiolated hydrogel allowing for robust capture and assessment of antibody concentration. For detecting galactosylation, the hydrogel may be linked with thiolated sugars and their corresponding lectins, which enables antibody capture based on glycan pattern. Importantly, the present disclosure provides linear assessment of total antibody concentration over an industrially relevant range and the selective capture and quantification of antibodies with terminal β-galactose glycans. Remarkably, the present sensors may be reused after surface regeneration using a low pH buffer. The present sensors with functionalized interfaces offer advantages in their simplicity, rapid assembly, connectivity to electronics, and reusability. As they assemble directly onto electrodes that also serve as I/O registers, the present sensors, devices, and systems may be incorporated into diagnostic platforms including those employed in manufacturing settings.

### EXAMPLES

This Example describes the development of sensor interfaces for titer and N-linked galactosylation detection that produce electrochemical outputs from electrodes upon which the sensing domains are also electroassembled. Unlike traditional analytical chemistry methods, our interfaces couple electrochemical techniques with biological molecular recognition elements. As illustrated in FIG. 1a, the interfaces are based on a thiolated polyethylene glycol (PEG) hydrogel film that is rapidly assembled by simply biasing an electrode that is immersed in a solution of thiolated PEG and a redox mediator, ferrocene (Fc), at pH 7 for 1 minute. The film thickness can be modified based on the time over which the electroassembly takes place. After electrodeposition, the hydrogel is submerged in Fc solution and further oxidized for 2 minutes in order to “activate” sulfhydryl groups to reactive sulfenic acid groups (SOH as indicated). The “activated” hydrogels are then able to be linked with either thiolated sugars (for assembly of glycan-recognizing lectins) or a newly developed cysteine-tagged protein G. The sulfenic acid groups of the PEG will spontaneously form disulfide linkages with thiols of sugars or exposed cysteine residues cloned into proteins at their termini. In this way, molecular recognition elements are covalently grafted onto the PEG, which in turn, is electroassembled directly onto the sensing electrodes. PEG is an ideal material for the interfaces as it enables the covalent linkage of the biological recognition elements and it provides anti-fouling properties that reduce non-specific binding.

For titer detection, the cysteinylated protein G construct is layered onto the PEG, where it enables total antibody titer detection via binding interactions with the antibody's fragment crystallizable (Fc) region. For glycosylation detection, instead of assembling the cysteinylated protein G, thiolated sugar groups (e.g., β-galactose, Galβ) occupy many of the open thiol-PEG binding sites so that the sugar groups act as a lectin bait. Then, sugar-specific lectins (e.g., Ricinus communis agglutinin I, RCA120) are layered on top of the galactosylated and thiolated PEG hydrogel. These surfaces then provide selective capture of antibodies based on traditional lectin-glycan binding. Further, when coupled with a protein G conjugated to horseradish peroxidase reporter (protein G:HRP), these interfaces enable near real time electrochemical outputs, based upon the electron transfer mechanism between HRP, ferrocene, and hydrogen peroxide (H₂O₂) as shown in FIG. 1b. As depicted, the same electrodes used to assemble the sensing components are used for electrochemical detection of the antibodies. As such, there are many advantages associated with the coupling of electroassembly and electrochemical readouts, a most simple example being that the size of the electrode can be used to vary the sensor binding capacity and signal strength. The methodology is simple, portable, inexpensive and can provide fast, quantifiable results for a variety of applications, including bioprocessing applications, noted here.

Materials. 4-Arm PEG thiol was purchased from JenKam (Plano, TX). RCA120 and fluorescein labelled conjugate (FITC RCA120) were purchased from Vector Laboratories (Burlingame, CA). 2-Thioethyl β-Galactopyranoside (thiolated Galβ) was purchased from Sussex Research Laboratories (Ottawa, ON Canada). Immunoglobulin (IgG) from human serum, SILU Lite SigmaMAb, and protein G:HRP conjugate were purchased from Millipore Sigma (Burlington, MA). 1,1′-Ferrocenedimethanol (Fc) was purchased from Santa Cruz Biotechnology (Dallas, TX). Dulbecco's Modified Eagle medium, high glucose, HEPES, no phenol red (DMEM) was purchased from Thermo Fisher Scientific (Waltham, MA).

PEG electrodeposition. A mixture of Fe (5 mM) and PEG thiol (50 mg/mL) was first prepared in phosphate buffer (0.1 M, pH 7.0). The surface of a 2 mm diameter gold standard electrode (working electrode) was fully immersed in the solution along with a platinum wire (counter electrode) and an Ag/AgCl reference electrode. PEG electrodeposition occurred for 1 minute at a constant potential of 0.4 V. After PEG hydrogel formation, remaining sulfhydryl groups were “activated” to sulfenic acids by immersing the surface in a solution of Fc (5 mM). A constant voltage of 0.4 V was applied for 2 minutes to ensure maximal sulfenic acid group formation on the surface of the hydrogel. Hydrogel electrodeposition and sulfhydryl “activation” were performed individually for each electrode preparation.

Assembly of interfaces. For the titer detection interface, the PEG-coated electrodes were individually immersed in cysteinylated protein G (250 μg/mL in 0.1 M PBS, pH 7.4) overnight. After 3 rinses with wash buffer (0.1 M PBS, 0.05% Tween-20, pH 7.4), the electrodes were sequentially immersed in IgG (diluted in 0.1 M PBS, 0.05% Tween-20, pH 7.4) and protein G:HRP (0.1 g/L in 0.1 M PBS, pH 7.4) for 1 hour at room temperature, respectively. IgG concentrations were confirmed by protein A₂₈₀ measurements prior to incubation using a NanoDrop 2000 spectrophotometer (Thermo Fisher Scientific). Between each immersion, the electrode was washed 3 times in wash buffer. For the fluorescence microscopy study, FITC cysteinylated protein G (250 μg/mL in 0.1 M PBS, pH 7.4) was used. Prior to use, cysteinylated protein G was FITC-labelled using the FluoReporter FITC labelling kit (Invitrogen; Waltham, MA) following instructions from the manufacturer. For the galactosylation detection interface, the PEG-coated electrodes were individually immersed in thiolated Galβ (50 μg/mL in 0.1 M PBS, pH 7.4) overnight at room temperature. After 3 rinses with wash buffer, the electrodes were sequentially immersed in RCA120 (0.5 mg/mL in 0.1 M PBS, pH 7.4), antibody glycoforms (1 mg/mL, diluted with 0.1 M PBS, pH 7.4), and protein G:HRP (0.1 g/L) for 1 hour at room temperature, respectively. RCA120 and antibody glycoforms concentrations were confirmed prior to incubation with protein A₂₈₀ measurements. For the fluorescence microscopy study, FITC RCA120 was used.

Electrochemical and fluorescence evidence of interface assemblies. Cyclic voltammetry (CV) and electrochemical impedance spectroscopy (EIS) measurements were taken using a CHI6273c electrochemical analyzer (CH Instruments; Austin, TX). The measurements were performed with a 3 electrode system containing bare gold or interface assembled electrodes (working electrode), a Pt wire (counter electrode), and a Ag/AgCl reference electrode, which were immersed in a phosphate buffered solution (0.1 M, pH 7.0) containing K₃Fe(CN)₆/K₄Fe(CN)₆ (1 mM). CV scans were performed from 0 to 0.5 V at a scan rate of 0.1 V/s. EIS measurements were taken over a frequency range of 100 kHz to 1 Hz at a potential that was the average of the reduction and oxidation peak potentials. Fluorescence microscopy images were then taken with an upright microscope with exposures of 90 ms (titer detection interface) and 50 ms (galactosylation detection interface).

Evaluation of hydrogel and interface reproducibility. PEG hydrogels were electrodeposited and activated as described herein. Charge profiles were measured during hydrogel electrodeposition. After electrodeposition and “activation,” CV scans were performed from 0 to 0.5 Vat a scan rate of 0.5 V/s in a solution of Fc (0.5 mM, diluted in phosphate buffer). For interface reproducibility, titer and galactosylation interfaces were individually assembled with FITC cysteinylated protein G (250 μg/mL) and FITC RCA120 (500 μg/mL) as described herein. Fluorescence microscopy images of the individual electrodes were taken with an upright microscope using exposure times of 20 ms (galactosylation detection) and 400 ms (titer detection). ImageJ was used to calculate the mean grey intensity values of the images.

Titer and galactosylation detection interface measurements. Measurements of the current response from the interfaces were performed with the 3-electrode set-up (same as above) using a CHI1040C electrochemical analyzer (CH Instruments). The electrodes were immersed in buffered solution (0.1 M phosphate, pH 7.0) containing Fc (0.5 mM) which was mechanically stirred at a constant rate with an applied potential of 0 V. To achieve a response from the interfaces, 25 μM H₂O₂ was added to the mixture and the increase in current from the baseline was recorded. The lower limit of detection for the titer detection interface was calculated as previously described.

Evaluation with culture media. Titer and galactosylation detection interfaces were assembled as described herein. Titer detection interfaces were incubated in IgG from human serum (0-1 g/L) that had been diluted with 0.1 M PBS or with DMEM. Galactosylation detection interfaces were incubated in SigmaMAb (1 g/L) diluted with 0.1 M PBS or with DMEM. This antibody standard was chosen as it has a known concentration of galactosylated IgG. Interfaces were then incubated in protein G:HRP (0.1 g/L). All incubations occurred at room temperature for 1 hour. Between incubations, interfaces were rinsed with wash buffer 3 times. Current measurements were taken as described herein.

Interface regeneration studies. To evaluate regeneration, the detection interfaces were assembled with IgG from human serum (2 g/L for titer detection, 1 g/L for galactosylation detection) and protein G:HRP (0.1 g/L) and measurements of the current response were taken using the conditions as herein. The interfaces were then washed with 0.1 M acetic acid, 1 M sodium chloride, pH 2.8 (3 washes, 15 minutes each) in order to strip IgG and protein G:HRP from the surfaces, leaving only the detection proteins (i.e. protein G or RCA120) conjugated to PEG surface. Current measurements were taken to confirm that IgG and protein G:HRP were removed. The interfaces were once again re-assembled with IgG and protein G:HRP. Current measurements were taken to confirm the re-binding of IgG and protein G:HRP to the interface surfaces.

### Example 1. Design, Expression, and Characterization of Cysteinylated Protein G

Protein G is a bacterial protein that possesses high binding affinity for the Fc domain of human immunoglobulin G (IgG). Here, protein G was used for the titer detection interface as it has been well-established that it can bind the most commonly used IgG subclasses for therapeutics development, IgG1 and IgG2, in cell culture broth. Protein G:antibody methodology was previously used for assembly of enzymes and cells onto various substrates. To incorporate protein G into this assembly and detection scheme, 5 cysteine codons were introduced into the C-terminus of the structural gene and expressed and purified the protein from E. coli. Because it is expressed in E. coli, the protein G was not glycosylated, which could otherwise confound subsequent antibody binding. The extra cysteine residues, in effect, become an activatable “pro-tag” and may be electroassembled based on the formation of a sulfenic acid enabled disulfide bond between the PEG and the protein G. In this example, the sulfenic acid of the PEG and the sulfhydryl of the cysteine residues enable covalent coupling.

Cells were cultured in LB media and induced with IPTG at OD₆₀₀˜0.4. The cells were centrifuged after they were grown overnight at room temperature. The cell pellets were then resuspended in lysis buffer, sonicated for 10 minutes, and then centrifuged to remove cell debris. The N-terminal His-tagged protein G was then purified using immobilized metal affinity chromatography (IMAC). The purified protein was confirmed by western blot and then used for assembly onto the thiolated PEG previously electroassembled onto gold electrodes.

The bacteria strains and plasmids used in this study are listed in Table 1. The construct for Cys5-tagged protein G is depicted in FIG. 9a. The sequence for protein G was preceded by six His residues at the amino terminus, followed by 5-cysteine residues located at the carboxyl terminus. The genes for protein G2-Cys were prepared by PCR amplification of a protein G template (pETE72G3) using primers listed in Table 2. The PCR product is then purified through gel electrophoresis and further inserted into pET200 vector via the Champion™ pET200 Directional TOPO® Expression Kit (Invitrogen; Waltham, MA). The derived plasmid that holds the protein of interest was confirmed by Sanger sequencing (Genewiz; South Plainfield, NJ) and subsequently transformed into BL21 Star™ (DE3) One Shot® E. coli for protein expression. The protein of interest was overexpressed under 1 mM IPTG induction as cell densities were grown at OD600=0.4-0.6 under 37° C. The cells were harvested after overnight induction at room temperature by centrifugation at 14000×g under 4° C. for 20 min. After lysis by BugBuster solution (Novagen; Madison, WI) at room temperature for 40 minutes, the soluble cell extracts were sonicated for 10 minutes and then centrifuged to remove cell debris. The cell extract was then loaded onto 5 mL HiTrap columns (GE Healthcare; Chicago, IL) and the bound target proteins were washed by binding buffer (20 mM phosphate, 0.5 M NaCl, 40 mM imidazole, pH 7.4) to remove non-specifically bound proteins. The proteins were eluted from the column by elution buffer (20 mM phosphate, 0.5 M NaCl, 0.5 M imidazole, pH 7.4). The purified proteins were then buffer exchanged using Slide-A-Lyzer dialysis cassettes (Thermo Fisher Scientific; Waltham, MA) into 0.1 M phosphate buffered saline, pH 7.4 for further experiments. The Coomassie stained polyacrylamide gel confirming cysteinylated protein G expression is shown in FIG. 9b.

TABLE 2

| | | Relevant |
|---|---|---|
| Name | Sequence | Description |
| FWD_ProteinG | CACCATGGGAGTTAAGATCCGCATGAC | Upstream |
| | (SEQ ID NO: 1) | primer for |
| | | cloning |
| | | Protein G |
| | | with a 5′- |
| | | CACC |
| | | sequence for |
| | | directional |
| | | cloning into |
| | | pET200 |
| REV_ProteinG- | CTAGCAACAACAACAACACAAGATCTTCGGGTCCATT | Downstream |
| Cys | TCCG (SEQ ID NO: 2) | primer for |
| | | cloning |
| | | Protein G |
| | | with 5- |
| | | cysteine tag |
| | | from pET- |
| | | E72G3 |

### Example 2 Assembly and Characterization of the Interfaces

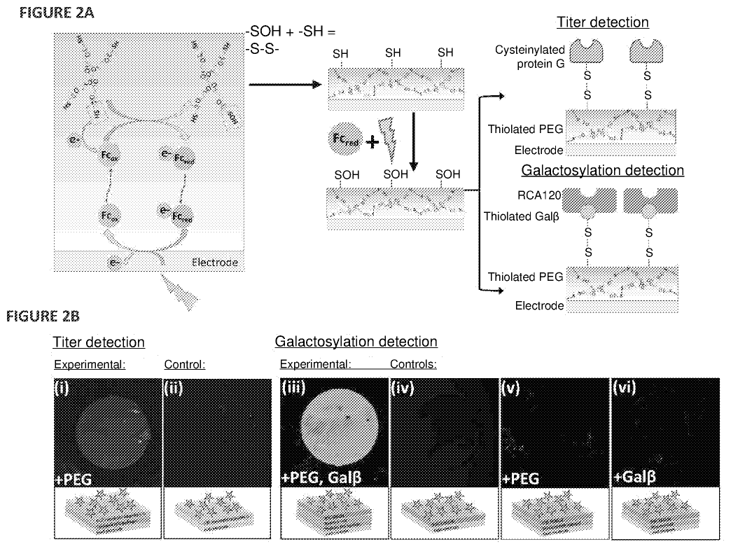

For both titer and galactosylation detection, a PEG hydrogel serves as the initial layer of the interfaces. FIG. 2a provides an illustration detailing the proposed mechanism for thiolated PEG electrodeposition. An oxidative potential (+0.4 V, 1 minute) is first applied to a bare gold electrode that is immersed in a mixture of Fc, a redox mediator, and 4-arm thiolated PEG. Fc near the surface of the electrode will become oxidized, diffuse away from the electrode surface, and oxidize the free thiol groups of PEG, converting them to sulfenic acid groups. Sulfenic acid groups will react with nearby thiol groups attached to PEG, thus establishing inter-molecular bonds allowing a hydrogel to form. The thiolated PEG directly assembles onto gold through the sulfur-gold interactions commonly used for templated assembly on gold. Mediated electroassembly using Fc is advantageous as it enables a level of control over the oxidation of the thiol groups of PEG, which allows for a significant level of consistency in the production of the hydrogels. That is, the electrodeposition process serves to “activate” the thiolated PEG in that the sulfenic acid groups will spontaneously bond with sulfhydryl groups forming covalent disulfide linkages. After PEG electrodeposition, the hydrogel is submerged in Fc solution and further oxidized for 2 minutes in order to “activate” the hydrogel (i.e. oxidize sulfhydryl groups to reactive sulfenic acid groups) to enable covalent bonding of the molecular recognition elements. For titer detection, the PEG modified electrode is then immersed in a solution of cysteinylated protein G enabling its assembly through disulfide bond formation between the cysteine tag of protein G and the sulfenic acid groups on the PEG hydrogel (upper path, FIG. 2a). The subsequent topmost protein G layer serves as the detection interface for IgG. For galactosylation detection, the electroactivated thiolated PEG surface can be immersed in a solution of thiolated Galβ, which will covalently bond with the available sulfenic acid groups on the surface of the hydrogel (lower path, FIG. 2a). Subsequently, immersion in a solution containing RCA120, a Galβ binding lectin, allows for bio-specific binding between RCA120 and thiolated Galβ, thus forming the galactose-detection surface of the interface.

In FIG. 2b, fluorescence microscopy was used to qualitatively characterize layer formation for both interfaces. FITC cysteinylated protein G and FITC RCA120 were used to visualize the binding of the proteins to the interfaces. For the titer detection interface, the presence of a PEG film was needed to establish fluorescence upon protein G capture. That is, there was no fluorescence in the absence of PEG (layering the assemblies onto bare gold). For the galactosylation detection interface, (iii) a strong fluorescence signal was observed only when all the interface layers were assembled. Controls, which included: (iv) bare gold; (v) an interface with no thiolated Galβ; (vi) and an interface with no thiolated PEG, were all weakly fluorescent, as expected. That is, when a component of the completely functional interface was not present (i.e. PEG or thiolated Galβ), there was essentially no FITC RCA120 binding. It is important to note that for all interfaces, the electrodeposition of PEG is localized on the pattern of the electrode, allowing for the interface to form only on the gold surface. This is an important attribute of the present process as electrochemical outputs (later) will be confined to activity that is localized directly onto the electrodes. Again, this is a feature of electroassembling functional components such as proteins, cells, and other molecules that are otherwise difficult to array with great spatial resolution. Because of this, electrochemical systems can be designed, and outputs quantified based on electrode area. This makes for well-controlled microfluidic application of the methodology.

Importantly, the data support the conclusion that the interfaces were successfully assembled when layers were sequentially formed, and all necessary components were present. Moreover, these data show that the thiolated PEG serves an important function as it provides a great number of binding sites (i.e. thiols) for protein assembly, enabling the interfaces' detection capabilities. In other work, it was shown that the electroactivated hydrogel properties could be extended away from the electrode (normal to the surface) the longer the voltage was applied, demonstrating programmable control of hydrogel chemistry. While not shown here, it is expected that the number of sulfenic acid residues available for coupling could be electronically programmed.

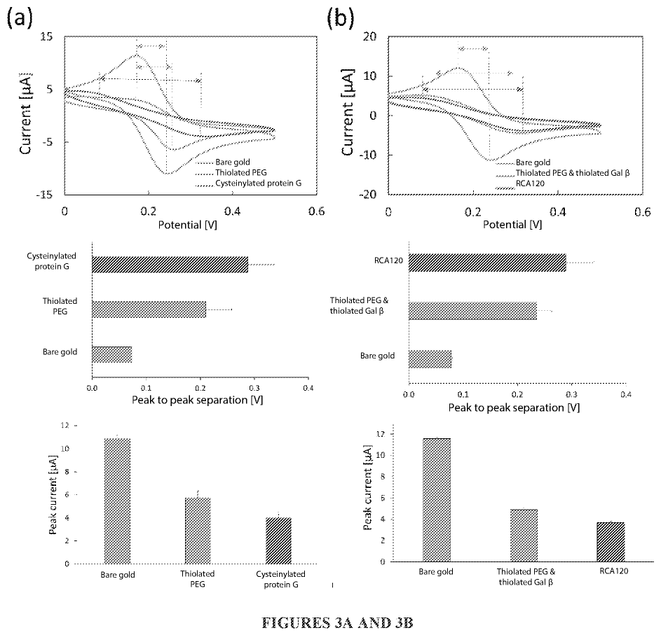

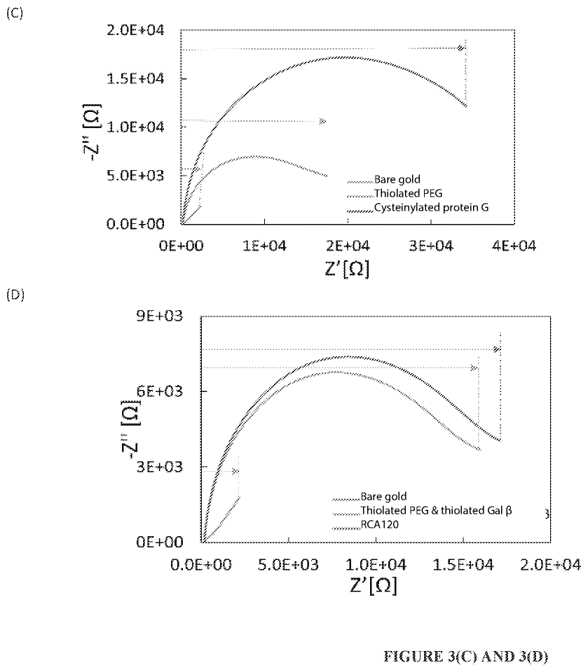

Next, cyclic voltammetry (CV) and electrochemical impedance spectroscopy (EIS) were used as an orthogonal means to provide physical evidence of interface assembly using redox probe, Fe(CN)₆^{3-/4-}. For CV measurements, bare gold electrodes for both interfaces had well-defined, reversible oxidation and reduction peaks, as shown in FIGS. 3a-3b. When the surfaces were electrodeposited with PEG, there was a clear reduction in both peak currents and an increase in the oxidation to reduction peak separation, as evidenced in the CV profiles and corresponding bar graphs. This indicates that electron transfer at the electrode surface was significantly decreased as a result of PEG hydrogel formation. For both interfaces, once the subsequent protein layer was assembled (i.e. cysteinylated protein G or RCA120), the peak current responses decreased further, and the peak-to-peak separation became larger, indicating that both proteins were successfully bound to the PEG-modified surfaces. EIS was also performed as it can sensitively detect changes in impedance through alterations in the surface charge of the assembled interface layers. The Nyquist plots of the titer and galactosylation detection interfaces are shown in FIGS. 3c-3d, respectively. At higher frequencies, the spectra have a semi-circular area that is related to charge transfer resistance (RcT) and at lower frequencies, they contain a linear portion which is related to diffusion processes. For the bare gold electrodes, the plots are essentially linear, indicating that there is minimal resistance to charge transfer. The diameter of the semi-circular area of the spectra becomes larger as the subsequent protein layers are formed on the surfaces (illustrated by the dotted arrows), indicating that RcT has increased due to further impedance of electron transfer. In both cases, CV and EIS measurements reinforce a conclusion that the interface layers were successfully assembled onto the electrode surfaces.

### Example 3. Reproducibility of Hydrogel and Interface Formation

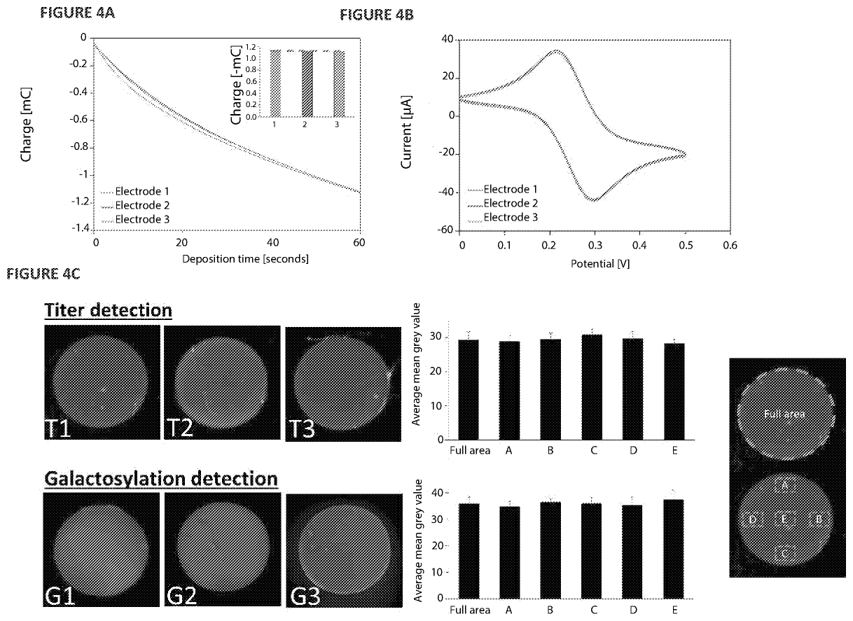

Next, the uniformity and reproducibility of hydrogel electrodeposition and interface assembly was characterized. Charge profile overtime throughout individual hydrogel electrodepositions was monitored for 3 separate electrodes. As shown in FIG. 4a, the charge is directly correlated to the number of sulfhydryl groups of PEG that are oxidized to sulfenic acid groups (i.e. electrons exchanged). Across all 3 hydrogel electrodepositions, the charge profiles were nearly identical (as depicted in the bar graph insert, the average final charge transfer was 1.1 mC+/−0.004). The data indicates that the number of sulfenic acid groups formed during deposition was identical and after considering the substantial reactivity of sulfenic acid it suggests that the degree of crosslinking among the hydrogels was also similar.

The consistency of the current measurements across different hydrogel preparations was also tested. Individually electrodeposited and “activated” hydrogels were immersed in a solution of 0.5 mM Fc and CV scans were performed. As shown in FIG. 4b, the CV scans of the 3 electrodes appear nearly superimposed, indicating that the current measurements are consistent for individually prepared hydrogels (i.e. electron exchange between Fe and the gold electrode was not impacted). To provide quantitative measure, the average oxidative (Q^{Ox}) and reductive (Q^{Red}) charge transferred were calculated from the CV profiles and the values were found to be consistent (Q^{Ox}=2247 ρC+/−42; Q^{Red}=1327 μC+/−48).

Lastly, fluorescence microscopy was used to gauge whether binding of the molecular recognition elements to individually assembled titer and galactosylation interfaces was also consistent. This is important in order to ensure that the interfaces provide for reproducible measurements. Electrodes were individually electrodeposited and “activated” as described herein. The hydrogels were then incubated in either FITC-labeled cysteinylated protein G (250 μg/mL) orthiolated Galβ (50 μg/mL) and FITC-labeled RCA120 (500 μg/mL). Fluorescence microscopy images were taken, and the fluorescence intensity of the images was analyzed using ImageJ. As shown in FIG. 4c, both the full circular area and 5 windows within each circular electrode were analyzed. The bar graphs represent the average intensities of the electrodes (Tl to T3 or G 1 to G3) for either the full electrode area or smaller areas from within the electrodes (A-E). For both interfaces, the variation in the intensity across the individual electrodes (error bars depict the standard deviation) was minimal, indicating that binding of molecular recognition elements to the hydrogels was remarkably consistent across separate electrode preparations.

### Example 4 Detection of Antibody Titer

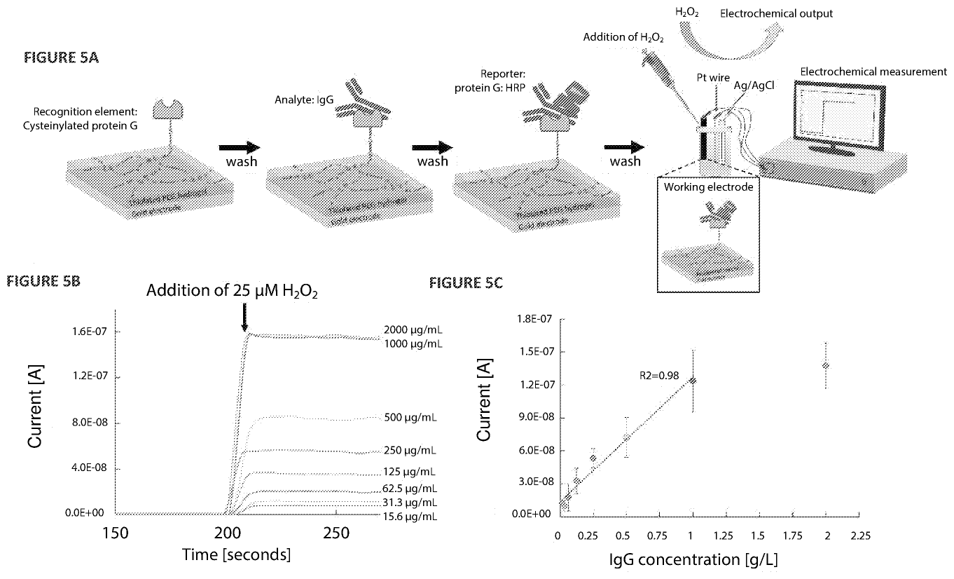

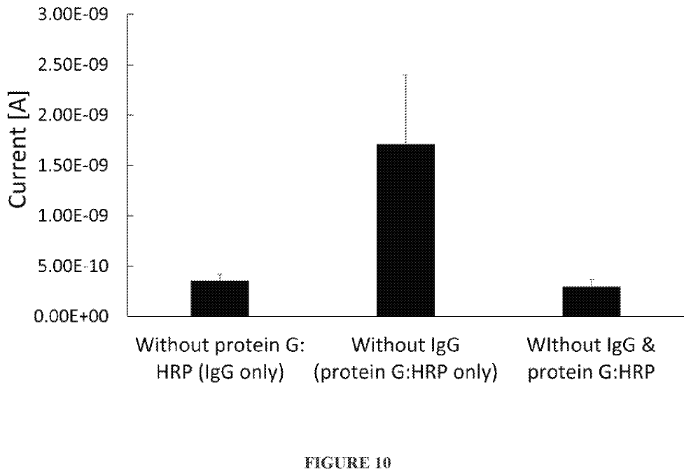

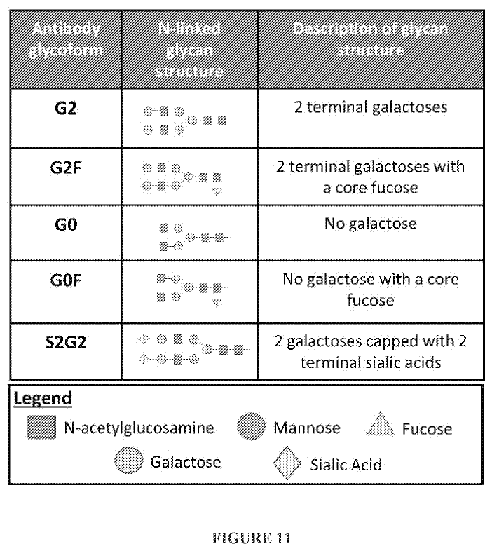

The ability of the cysteinylated protein G interface to detect antibodies was evaluated. The experimental process and set-up is illustrated in FIG. 5a. The assembled cysteinylated protein G interfaces were incubated with serially diluted concentrations of human IgG, washed with buffer containing 0.05% Tween-20 to remove any non-specific binding, and then immersed in protein G:HRP to form a sandwich configuration. It is important to note that IgG has 2 protein G binding sites on either side of its Fc portion (located in the hinge region that connects the CH2 and CH3 domains). This allows protein G to serve as both the recognition (cysteinylated protein G) and detection element (protein G:HRP). After many experiments, it was found that a 0.1 g/L concentration of protein G:HRP (diluted in PBS) was best to maximize the electrochemical responses of the assembled 3.1 mm² interfaces while minimizing responses from non-specific binding. The cysteinylated protein G-IgG-protein G:HRP interfaces were then submerged in a stirred solution containing 0.5 mM Fc (diluted in phosphate buffer) and with an applied potential of 0 V. The solutions were equilibrated for 200 seconds and then 25 μM H₂O₂ was spiked into the solutions to achieve the current responses from the cysteinylated protein G-IgG-protein G:HRP interfaces, shown in FIG. 5b. In an analogous manner to the studies with protein G:HRP, earlier studies were performed with varied amounts of H₂O₂ and it was found that 25 uM was optimal for the conditions tested. The results similarly indicate that response time is very rapid. After the initial transient, the current at all IgG concentrations reached a steady value within 2 sec, and importantly, reached a very steady value that was maintained for an additional 2 minutes. The current appeared to be related to the concentration of IgG in the incubation buffer. In FIG. 5c, it was found that when the interfaces were incubated with IgG concentrations ranging from 15.6-1000 μg/mL, the average of the peak current response linearly increased with the concentration of IgG (R2=0.98). Importantly, this linear range for the interface is appropriate for many antibody therapeutics produced in mammalian cell lines such as Chinese hamster ovary (CHO) cells. That is, the response was observed to saturate above a concentration of 1000 μg/mL of IgG (note the 1000 μg/mL vs. the 2000 μg/mL responses). Using 3 times the standard deviation of the negative control, it was found that the lower limit of detection was 88 μg/mL. Notably, these results were from separate measurements across separate batches of individually assembled interfaces and their linearity demonstrates that the method is highly reproducible even when using different gold electrodes. The responses from the negative controls further show that non-specific binding from protein G:HRP was minimal (FIG. 10). As noted above, a low level of non-specific binding is likely attributed to the anti-fouling properties of PEG. Overall, these data support the interface's applicability for use in monitoring titer in support of antibody production and separation operations.

### Example 5 Detection of Antibody Galactosylation

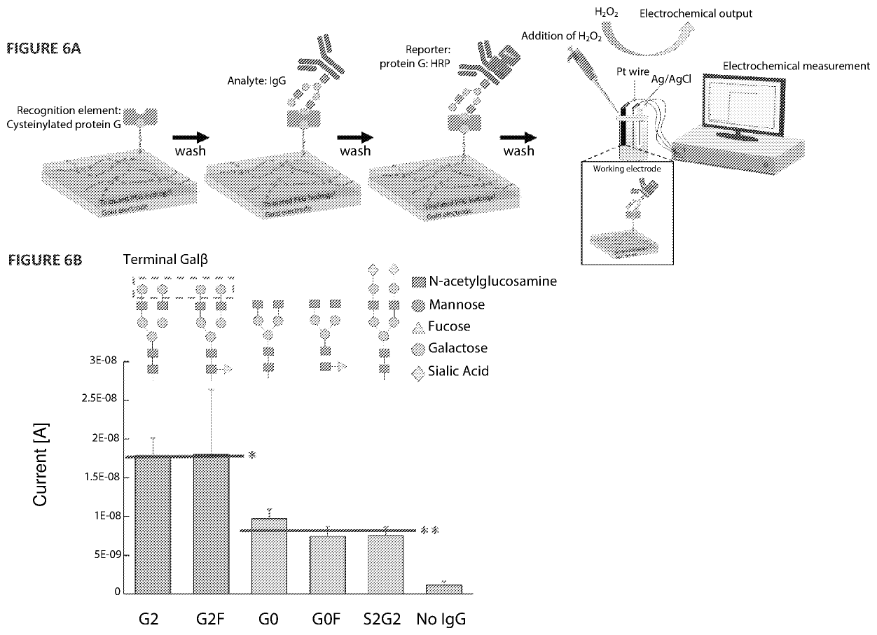

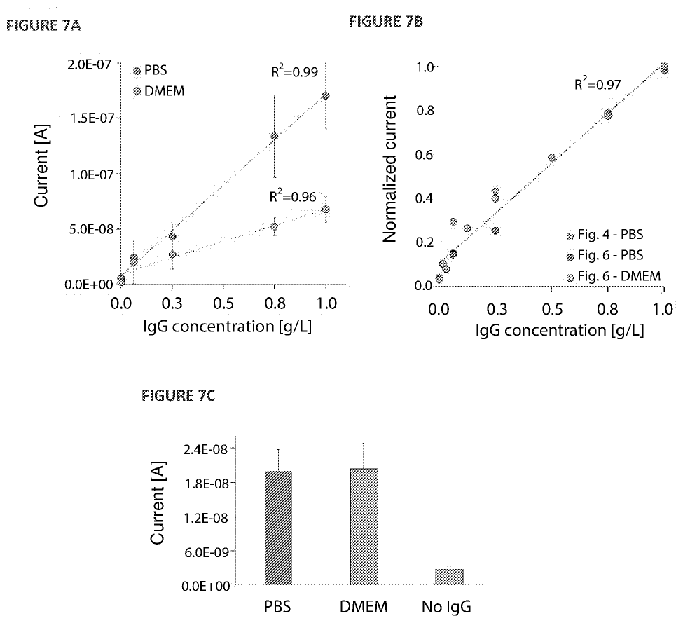

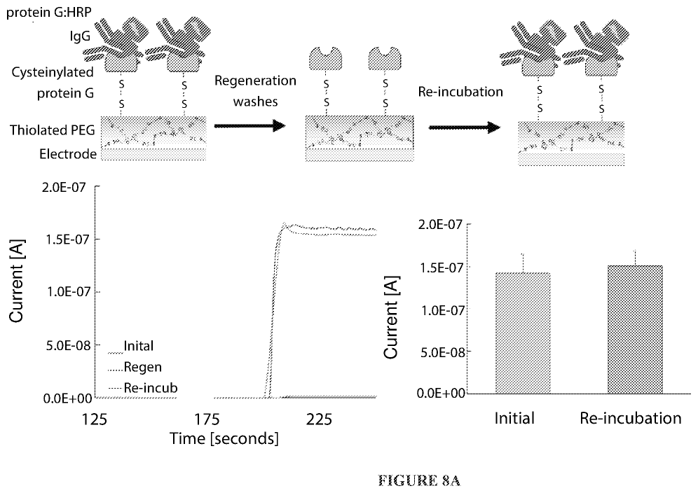

The selectivity of the galactosylation detection interface for Galβ terminating oligosaccharides was then evaluated. FIG. 6a depicts the experimental process and set-up, which was performed in an identical manner as the titer studies above. Solutions containing dilutions of 1 g/L of each respective antibody glycoform (i.e. antibodies with a defined N-linked glycan pattern) were incubated with the interfaces. The antibody glycoforms used to interrogate the selectivity of the interface had glycan patterns of G0, G0F, G2, G2F, and S2G2, which are described in FIG. 11 and depicted in FIG. 6b.

These represent many of the common glycan structures of antibody therapeutics, which are of the complex diantennary type that contain variable additions of fucose, galactose, and sialic acid. These antibody glycoforms were previously developed. Galβ was chosen as a model terminal glycan group for interface detection as it is one of the most predominant terminal N-linked glycan modifications. That is, several interfaces were examined with the test case being RCA120 assembled onto PEG with thiolated Galβ. Controls with different interface assemblies (no thiolated PEG, no thiolated Galβ) and different IgG (altered glycan patterns, including sialic acid capped galactose) were also evaluated. Lectin RCA120 was used for galactosylation detection as: (i) it has been shown to have specific binding to Galβ and none of the other glycans and, (ii) it contains two subunits that have binding affinity for Galβ terminating oligosaccharides, making it ideal for interface assembly as one binding site is already occupied by the thiolated Galβ. In order to maintain consistency, concentrations of 1 g/L IgG, 0.1 g/L protein G:HRP, and 25 μM H₂O₂ were selected as these conditions helped to maintain linearity for the titer detection interface.

As shown in FIG. 6b, the lectin-containing interface shows the highest current for the antibody glycoforms that contain terminal Galβ (i.e. G2 and G2F). For antibody glycoforms not containing Galβ (i.e. G0 and G0F) or when capped by a different glycan, such as sialic acid (i.e. S2G2), the current responses were significantly lower and relatively similar to one another, indicating that the interface selectively recognizes glycan structures that contain terminal Galβ. That is, measurements for galactosylated antibodies, G2 and G2F, were statistically different from the non-galactosylated antibodies, G0, G0F, and S2G2 (p<0.005). Similar to the titer detection interface, there was minimal response for the controls with no assembled IgG. This confirms that there was minimal contribution to the response from non-specific binding of protein G:HRP. This was tested using other thiolated sugars, RCA120, and galactose capped IgG and there was essentially no signal (not shown here). As this methodology employs a thiolated sugar and its corresponding lectin, the results suggest that the functionalized thiolated PEG interface could serve as a platform for other glycan specific, lectin-based detection interfaces for glycoprofiling purposes. Multiple interfaces that detect different glycan groups might then be used in parallel to give a rapid, high-throughput analysis of the glycan structures that are present on the antibodies. Lectin arrays using different assembly methodologies are commercially available, often employing fluorescence outputs on spotted membranes or microscope slides. Detection limits and selectivity of the interface may be determined for antibody glycoforms that contain varying numbers of galactose (i.e. G1, G1F) and sialic acid moieties (i.e. S1G1, S1G2).

### Example 6 Evaluation with Culture Media

To expand on the applicability of the interfaces for upstream bioprocessing, the interfaces were characterized with a representative sample from an upstream setting. The interfaces were evaluated with IgG spiked into mammalian cell culture media (i.e. Dulbecco's Modified Eagle medium, DMEM) to determine if media components affected the performance of the interfaces. Incubation and current measurement conditions using the interfaces were identical to those described herein, except that the interfaces were immersed in solutions of IgG that were spiked into either PBS with 0.05% Tween-20 or DMEM. Results are shown in FIGS. 7a-7c. Importantly, in FIG. 7a, linearity was re-established for the IgG spiked in PBS with 0.05% Tween-20, consistent with results in FIGS. 5a-5c. However, the average current values in FIGS. 7a-7c were uniformly higher than those in FIGS. 5a-5c even though identical experimental conditions were used. Interestingly, as film assembly is robust and reproducible, this suggests these differences were due to batch to batch differences in the preparations of cysteinylated protein G and/or protein G:HRP. Both of these were added to the interfaces as mg quantities and their activity might vary based on batch. A strong linear response was obtained for the IgG spiked DMEM samples, but with a lower slope. It was suggested that the apparent lower currents coincide with media components lowering the number of binding events between the protein G and the IgG. All data for antibody titer in FIG. 7b were superimposed by normalizing each dataset by the maximum current obtained for the 1 g/L incubations; importantly linearity was maintained.

Interestingly, negligible differences were found between measurements of IgG in PBS or DMEM for the galactosylation detection (FIG. 7b). As current responses from both samples were nearly identical, DMEM did not seem to impact the binding between RCA120 and galactosylated IgG. A hypothesis for these observations was provided. Overall, these data indicate that both of the interfaces can be used to evaluate IgG in samples of mammalian cell culture media.

### Example 7 Regeneration of Interfaces

Surface regeneration of the interfaces was investigated to test whether they could be used for more than a single measurement. After several experiments and in concordance with the literature, it was found that 0.1 M acetic acid and 1 M sodium chloride, pH 2.8 could be used as a regeneration buffer as low pH conditions are often used to effectively decouple many protein-antibody interactions. This regeneration buffer was used to remove IgG while retaining the cysteinylated protein G (for the titer detection interface) and the thiolated sugar and potentially, bound lectin (for the galactosylation detection interface). In this way, we retain the oxidized disulfide bonds and the functionalized hydrogel, but release product IgG.

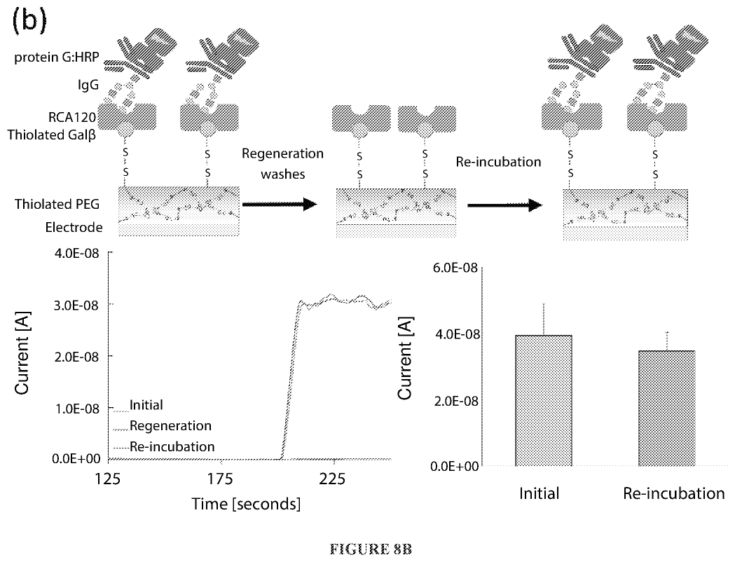

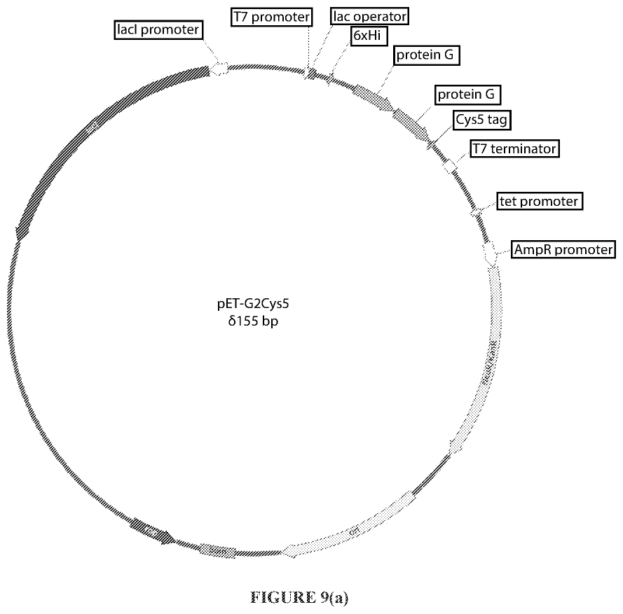

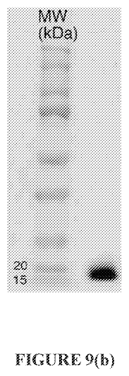

As depicted in the schematics (top) in FIGS. 8a-8b, after initial measurements of samples containing IgG (2 mg/mL IgG for titer detection, 1 mg/mL IgG for galactosylation detection) and 0.1 g/L protein G:HRP, the surfaces of the assembled titer and galactosylation detection interfaces were washed 3 times (15 minutes each) with the low pH regeneration buffer. Then, after the regeneration washes, additional control measurements (added H₂O₂) were made to ensure that the current responses were removed and that the surfaces had been successfully stripped of IgG and protein G:HRP. As seen in representative current responses (left) shown in FIGS. 8a-8b, the interface responses were entirely diminished suggesting that complete regeneration was achieved. Then, the interfaces were sequentially incubated with solutions containing IgG and protein G:HRP and repeat measurements were taken. After re-incubation using the same IgG concentrations, the responses returned to nearly identical signal strengths as the initial measurements. Interestingly, the dynamic responses were also unchanged. In all cases, the peroxide-mediated signal transfer was rapid (within seconds) and very stable. These data demonstrate that the interfaces could replicate the initial responses without incurring significant loss of activity. Both bar graphs (right) show that the absolute differences in response between the initial and re-incubation measurements for both the titer and galactosylation detection interfaces were 6.8% and 12%, respectively. Notably, the variations observed were both positive and negative in direction, suggesting there was no systematic error involved. Instead, deviations were likely due to random errors associated with the re-use protocol developed.

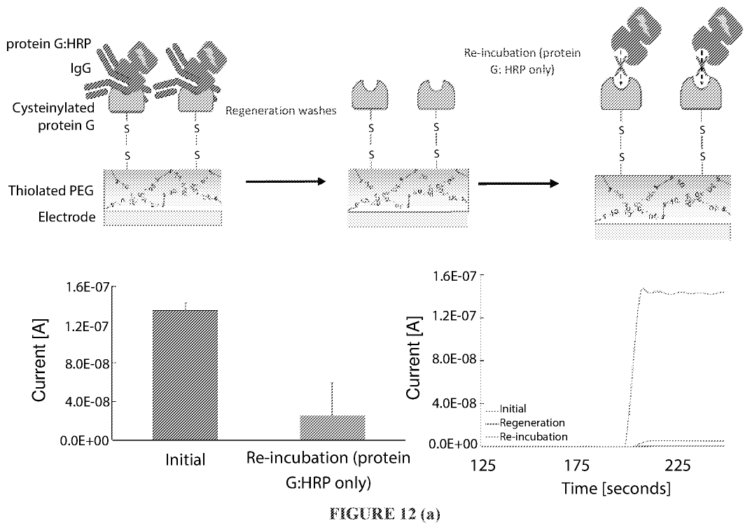

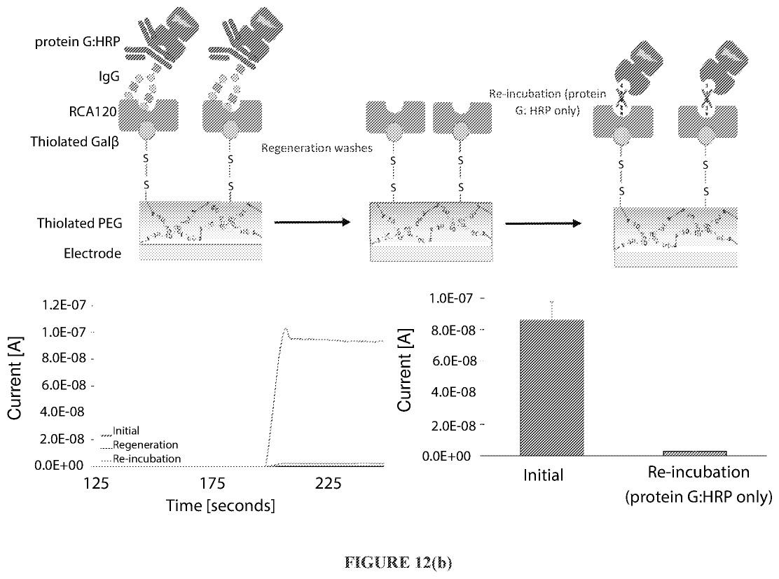

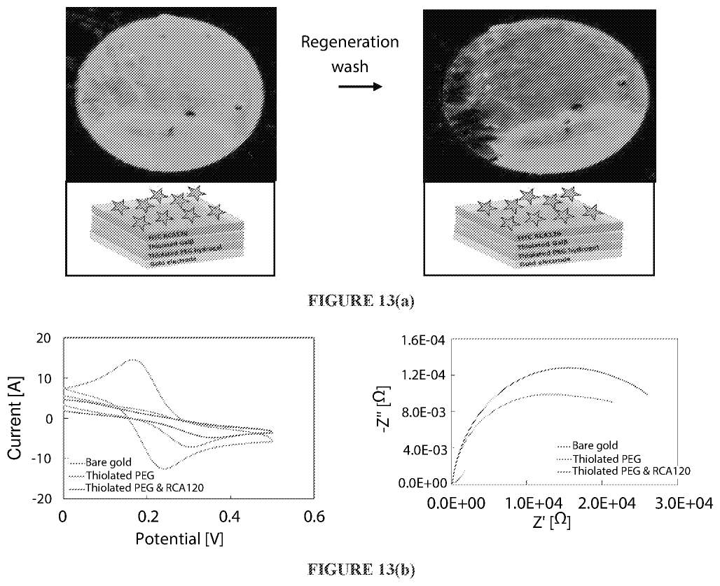

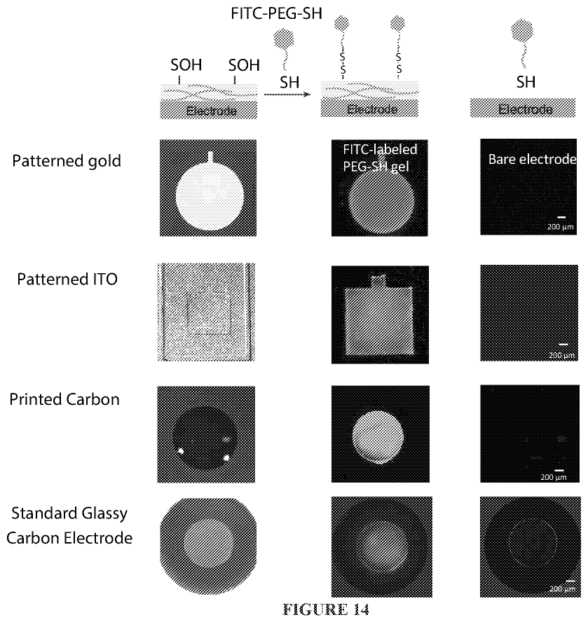

While these results were quite satisfactory, the regeneration process was further evaluated regarding the apparent discord between maximum currents obtained in DMEM and PBS noted above. In FIGS. 12a-12b, the interfaces were regenerated after an initial measurement and then, unlike in FIGS. 8a-8b, the surfaces were incubated with protein G:HRP only to confirm that bound IgG had been removed. The low current obtained confirmed its effective removal from the interfaces. That said, it was noted that during the regeneration and reuse of the galactosylation interface, subsequent addition of IgG indicated that the RCA120 lectin had been retained and was apparently not rate limiting in the analysis (FIG. 8b). This was unexpected given that the manner of binding of RCA120 to the thiolated Galβ on the hydrogel is non-covalent. To investigate further, fluorescence microscopy was used with FITC RCA120 after regenerating the surface (FIG. 13a). A strong fluorescent signal was retained (although, as expected, there was an observable loss compared to pre-wash) suggesting that a significant quantity of RCA120 remained bound to the interface. The surfaces were reevaluated with both CV and EIS. Results shown in FIG. 13b suggest a potential explanation; RCA120 was nonspecifically bound to the hydrogel through direct interactions with the thiolated PEG and thus was still attached to the surface after regeneration. The CV (FIG. 13b, left) and EIS (FIG. 13b, right) profiles were measured without the thiolated Galβ and there was a degree of binding of RCA120 to the hydrogel. These results might provide insight regarding the retention of galactose binding activity after regeneration, as noted above in FIGS. 7a-7c. The addition of DMEM did not decrease detection of galactosylation, while the titer measurement had dropped in half at the highest concentrations tested. It was hypothesized that media components in DMEM might have lowered the number of binding events between protein G and the IgG. However, since there were likely many RCA120 sites beyond those of the Galβ, such attenuation was not observed. Notably, the enhanced stability of the lectin assembly to the oxidized thiolated PEG was a welcome surprise.

The exploratory binding studies reinforced that the thiolated and functionalized PEG interfaces were robust and have the potential for multiple antibody-based measurements, potentially providing significant benefits to antibody development and production processes. Moreover, RCA120 binding to the thiolated PEG was not anticipated, but it is a welcome finding. The lifetime and the robustness of the interfaces after multiple regeneration cycles may be determined, and these interfaces may be integrated into a microfluidic device.

This Example provides novel thiolated PEG interfaces that are easily assembled onto gold electrodes. Owing to the abundance of thiol groups and the ease by which they can be oxidized or reduced, the interfaces offer the ability to be functionalized for bioprocess monitoring applications. The present disclosure demonstrates new formats for the detection of antibody titer as well as the glycosylation pattern on human IgG using a simple and rapid electrochemical reporting method. The platform may be amenable for a variety of thiolated sugars and lectins. This would expand its use beyond the galactosylation detection shown here, providing for a more high-throughput analysis of the glycan structures. Equally importantly, a cysteine-tagged protein G reagent was expressed and purified from E. coli (it has no glycosylation). This cysteine-tagged reagent, when coupled with the thiolated PEG interface, enables versatile functionalization of the surfaces in that antibody assembly and near real time monitoring of antibody titer is enabled. Further, both interfaces disclosed herein can be successfully regenerated for reuse. This helps in terms of cost savings and accelerated analysis. These interfaces and the ease by which they can be assembled may enable many such functionalized surfaces, especially those that integrate biological components within microelectronic devices. In biomanufacturing, rapid assessment of antibody titer and galactosylation should reduce both cost and time associated with the development and production processes.

It will be apparent to those skilled in the art that numerous changes and modifications can be made in the specific embodiments of the invention described above without departing from the scope of the invention. Accordingly, the whole of the foregoing description is to be interpreted in an illustrative and not in a limitative sense.

What is claimed is:

1. A sensor comprising:
an electrode comprising an electronically conductive surface;
a polymer layer deposited on the electronically conductive surface of the electrode; and
a biological recognition element attached to the polymer layer.

2. The sensor of claim 1, wherein the electronically conductive surface comprises a metal, a metal oxide, a conductive glass, and/or a conductive carbon material.

3. The sensor of any one of claims 1-2, wherein the electronically conductive surface comprises gold.

4. The sensor of any one of claims 1-3, wherein the electronically conductive surface is a patterned surface.

5. The sensor of any one of the preceding claims, wherein the polymer layer is electrodeposited on the electronically conductive surface of the electrode.

6. The sensor of any one of the preceding claims, wherein the polymer layer comprises a hydrogel.

7. The sensor of any one of the preceding claims, wherein the polymer layer comprises thiolated polyethylene glycol (PEG).

8. The sensor of any one of the preceding claims, wherein the biological recognition element is attached to the polymer layer by a disulfide bond (S—S).

9. The sensor of any one of the preceding claims, wherein the biological recognition element comprises a cysteine-tagged protein G or a thiolated sugar, optionally wherein the thiolated sugar is linked to a sugar-specific lectin.

10. The sensor of claim 9, wherein the biological recognition element comprises the thiolated sugar, and wherein the thiolated sugar is deposited on the polymer layer and the sugar-specific lectin is deposited on top of the thiolated sugar.

11. The sensor of claim 10, wherein the thiolated sugar comprises a thiolated saccharide, optionally wherein the thiolated saccharide is a thiolated monosaccharide.

12. A device comprising the sensor of any one of claims 1-11 and at least one additional component.

13. The device of claim 12, wherein the at least one additional component comprises a housing, a counter electrode, a reference electrode, a computer, a data storage unit, a controlling unit, a power supply, or combinations thereof.

14. The device of claim 12 or 13, comprising an array of a plurality of the sensors.

15. A method of preparing a sensor, comprising:
(a) contacting an electrode comprising an electronically conductive surface with a redox mediator and a polymer in a buffer solution;
(b) depositing the polymer onto the electronically conductive surface of the electrode to generate a polymer-coated electrode, wherein the polymer-coated electrode comprises a plurality of —SH groups; and
(c) applying to the polymer-coated electrode a biological recognition element.

16. The method of claim 15, wherein polymer in (a) comprises a plurality of —SH groups.

17. The method of any one of claims 15-16, wherein (b) comprises applying an electric potential to the electrode, thereby electrodepositing the polymer onto the electronically conductive surface of the electrode.

18. The method of any one of claims 15-17, wherein (b) comprises crosslinking the polymer, optionally wherein the crosslinked polymer forms a hydrogel.

19. The method of any one of claims 15-17, wherein (b) comprising
(b-i) depositing the polymer onto the electronically conductive surface of the electrode; optionally wherein the deposited polymer are crosslinked to form a hydrogel; and
(b-ii) attaching a plurality of thiolated linker molecules to the deposited polymers to generate the polymer-coated electrode.

20. The method of any one of claims 16-19, wherein (b) comprises crosslinking the polymer by forming a disulfide bond (S—S) between the —SH groups of the polymer.

21. The method of any one of claims 17-20, wherein (b) further comprises converting a plurality of —SH groups of the polymer-coated electrode to —SOH groups by the applied electric potential.

22. The method of any one of claims 15-21, wherein the biological recognition element is attached to the polymer-coated electrode by a disulfide bond (S—S).

23. The method of any one of claims 15-22, wherein the electronically conductive surface comprises gold.

24. The method of any one of claims 15-23 wherein the polymer comprises thiolated polyethylene glycol (PEG).

25. The method any one of claims 15-24, wherein the redox mediator comprises ferrocene or a derivative thereof.

26. The method of any one of claims 15-25, wherein step (c) comprises immersing the polymer-coated electrode in a solution comprising a cysteine-containing protein for a sufficient time to generate a protein-coated electrode.

27. The method of claim 26, wherein step (c) comprises
(i) immersing the polymer-coated electrode in a solution comprising a cysteine-tagged protein G for a sufficient time to generate a protein G-coated electrode, wherein the protein G-coated electrode is capable of determining antibody titers; or
(ii) immersing the polymer-coated electrode into a solution comprising thiolated sugar for a sufficient time to generate a sugar-coated electrode, and optionally subsequently immersing the sugar-coated electrode in a solution comprising a sugar-specific lectin for a sufficient time to generate a lectin-coated electrode, wherein the sugar-coated electrode and/or the lectin-coated electrode is capable of antibody capture.

28. A method of detecting and/or quantitating an analyte in a sample, comprising
(a) contacting the sample with the sensor of any one of claims 1-11 or the device of any one of claims 12-14, wherein the analyte binds to the sensor or the sensor of the device; and
(b) detecting the bound analyte from the sample on the sensor.

29. The method of claim 28, wherein the analyte is a protein, optionally wherein the protein is an antibody or fragment thereof, optionally wherein the antibody is an IgG antibody, optionally wherein the antibody is a glycosylated antibody.

30. The method of any one of claims 28-29, wherein the biological recognition element of the sensor comprises a cysteinylated protein G.

31. The method of any one of claims 28-29, wherein the biological recognition element of the sensor comprises a thiolated saccharide, optionally wherein the thiolated saccharide is a thiolated monosaccharide.

32. The method of claim 31, wherein the biological recognition element comprises a thiolated saccharide linked to a saccharide binding protein.

33. The method of claim 32, wherein the saccharide binding protein is a lectin, optionally wherein the lectin is RCA120.

34. The method of any one of claims 28-33, wherein detecting the attached analyte comprises measuring an electrochemical response of the electrode.

35. The method of any one of claims 29-34, wherein detecting the attached analyte comprises measuring an electrochemical response of the electrode in a redox cycling reaction.

36. The method of any one of claims 34-35, wherein measuring the electrochemical response of the electrode comprises measuring cyclic voltammetry (CV) and/or electrochemical impedance spectroscopy (EIS).

37. The method of any one of claims 28-36, where detecting the attached analyte comprises quantitating the attached analyte.

38. The method of any one of claims 28-37, further comprising removing the attached analyte following detection of the analyte to form a regenerated sensor.

39. A method of detecting and/or quantitating an antibody in a sample, comprising
(a) contacting the sample with the sensor of any one of claims 1-9, wherein the biological recognition element comprises a cysteine-tagged protein G, wherein the antibody in the sample binds to the sensor; and
(b) detecting the attached antibody.

40. A method of analyzing a glycosylated antibody in a sample, comprising
(a) contacting the sample with the sensor of any one of claims 1-11, wherein the biological recognition element comprises a thiolated sugar, optionally wherein the thiolated sugar is linked to a sugar-specific lectin, thereby attaching the glycosylated antibody in the sample to the sensor; and
(b) detecting the attached glycosylated antibody.

41. The method of claim 40, further comprising determining a glycan structure of the glycosylated antibody.

42. The sensor of any one of claims 1-11 or device of claims 12-14, wherein the cysteine-tagged protein G comprises SEQ ID NO:1.
